(19) United States
(12) Patent Application Publication
(10) Pub. No.: US 2025/0277271 A1
(43) Pub. Date: Sep. 4, 2025

(54) Methods of Assessing Smoldering Multiple Myeloma

(71) Applicant: TELO GENOMICS HOLDINGS CORP., Toronto (CA)

(72) Inventors: Sabine MAI, Winnipeg (CA); Sherif LOUIS, Toronto (CA); Hans KNECHT, Magog (CA)

(21) Appl. No.: 18/861,552

(22) PCT Filed: Nov. 16, 2023
(86) PCT No.: PCT/CA2023/051546

§ 371 Date: Oct. 29, 2024

(30) Foreign Application Priority Data
Nov. 16, 2022 (WO) PCT/CA2022/051694

(51) Int. Cl. C12Q 1/6886; C12Q 1/6841; G16B 15/10; G16B 25/00; G16B 40/20

(52) U.S. Cl. CPC C12Q 1/6886; C12Q 1/6841; G16B 15/10; G16B 25/00; G16B 40/20; C12Q 2600/112; C12Q 2600/118; C12Q 2600/158

(57) ABSTRACT

Provided are improved methods for prognosing a clinical outcome in a subject or diagnosing the subject with high-risk or stable smoldering multiple myeloma (SMM), comprising determining a 3D telomeres organization signature of a test sample from the subject, the test sample comprising plasma cells, applying a classification model to the 3D telomeres organization signature to obtain an output classification that is indicative of the clinical outcome or diagnosis of the subject. The classification model is trained to distinguish between stable SMM and high-risk SMM and consists of the telomere parameters: a) nuclear telomere distribution, a/c ratio, and total telomere length; b) a/c ratio and telomere numbers; c) a/c ratio and nuclear telomere distribution, or d) a/c ratio and telomere aggregates. Also provided are methods for treating a subject with high-risk or stable SMM.

### RELATED APPLICATIONS

The present application claims the benefit of priority of International Application no. PCT/CA2022/051694 filed on Nov. 16, 2022.

### FIELD

The present disclosure relates to methods of differentiating patients with smoldering multiple myeloma and more particularly to methods of differentiating patients with different risk profiles of smoldering multiple myeloma or differentiating patients from other related diseases based on three dimensional telomeric organization.

### BACKGROUND

Multiple myeloma (MM) is a B-cell malignancy characterized by extensive proliferation of plasma cells (PCs) in the bone marrow (BM) and abnormal increase of monoclonal immunoglobulins or M proteins [1]. This aberrant plasma cell proliferation leads to lytic bone lesions, hypercalcemia, kidney failure and severe anemia [1].

MM is the end stage of monoclonal gammopathy of undetermined significance (MGUS) and smoldering multiple myeloma (SMM) [2,3]. All cases of MM are preceded by MGUS or SMM [2,3]. MGUS patients have detectable monoclonal immunoglobulin in blood or urine (<3 g/dL) and clonal bone marrow plasma cells under 10%, but no evidence of end-organ damage or other symptoms. The risk of progression of MGUS to MM is about 1%-2% per year [4,5]. In SMM patients, the levels of M protein in the serum are higher than in MGUS (≥3 g/dL) and clonal bone marrow plasma cells greater than 10% but less than 60%, with patients still showing no symptoms and no laboratory signs of end-organ damage, but the risk of progression to MM is about 10% per year in the first 5 years, particularly in subjects with blood disorders family history, and/or genetic ethnic background [4,5, 38].

Despite the development of novel therapeutic strategies and a better understanding of MM biology, this disease remains incurable with a heterogeneous clinical course, where overall survival can range from a few months to over 10 years [6]. Therefore, the development of early reliable surrogate end-points for survival is necessary for better risk stratification, treatment individualization, and future incorporation of novel agents and combinations into the MM management. Among the many prognostic factors and several different risk stratification systems described for MM, the International Staging System (ISS), initially based on serum levels of both β2-microglobulin and albumin [7], has recently incorporated cytogenetic abnormalities and lactate dehydrogenase levels [7]. However, gene expression profile, plasma cell proliferative rate, extramedullary disease, initial presentation as plasma cell leukemia, age, performance status, PET-CT presentation, and comorbidities are also taken into consideration in the clinical perspective on treatment goals and management of MM patients [7].

The relatively modest rate of progression of MGUS to the full stage MM does not imply significant concern for managing MGUS patients. However, the progression of SMM patients to full stage MM has been a burden in the clinic for the past few decades, especially due to the absence of biomarkers that can predict the risk of the individual SMM patient to transition to full stage MM with high specificity [39 & 40].

Furthermore, several clinical studies have shown significant advantage in time-to-progression and overall survival of SMM patients if treated with chemotherapy [41]. Nevertheless, with approximately 90% of the SMM patients presenting a stable form of the SMM disease, the decision to treat all SMM patients with chemotherapy is far from being adopted in standard clinical practice, given the significant side effects associated with these therapies.

In recent years, several approaches have attempted to stratify SMM patients into risk groups including multiple risk stratification models based on traditional clinical risk factors. These models attempted to classify patients into a group with over 50% chance, and a group with under 50% chance, of transitioning to full stage MM within 2 years; however, satisfactory sensitivity was not achieved [39 & 40]. To date, identifying high risk SMM patients that could benefit from chemotherapy or other interventions and confirming the disease stability in low risk SMM patients remain critical clinical unmet needs in the management of MM [39, 40 & 41].

A prominent feature observed in MM cells is a dynamic genomic instability and complexity, which increases with subsequent acquisition of additional genetic abnormalities [8]. The cell-to-cell heterogeneity and presence of multiple subclones affect both prognostic stratification and therapeutic approaches [9]. This intraclonal diversity, where different clones are present at diagnosis and during disease evolution, promotes survival advantage of individual clones upon treatment, selection of minor pre-existing or novel clones, and disease progression [9-12].

Methods for risk stratification or differentiating high risk SMM from low risk SMM are desirable.

### SUMMARY

The present inventors have identified improved diagnostic and prognostic classification models for subjects having smoldering multiple myeloma (SMM) based on 3D telomere analysis of plasma cells. For example, it is demonstrated that SMM patients can be classified as having stable SMM not likely to progress to multiple myeloma (MM) within 2 years or as having high-risk SMM likely to progress to MM within 2 years based on their telomere signature.

Accordingly in an aspect, the disclosure includes a method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; and
optionally providing the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with stable SMM is not likely to progress to MM within 2 years.

In one embodiment, test sample is a bone marrow sample or a blood sample, optionally a diagnostic bone marrow biopsy sample or a peripheral liquid biopsy blood sample.

In one embodiment, the prognosis or the diagnosis is provided to the subject or the subject's medical professional, optionally at time of SMM diagnosis.

In one embodiment, the assaying comprising:
labelling nuclei of the plurality of the plasma cells with a fluorescent nuclear stain or probe, optionally wherein the fluorescent nuclear stain is 4′,6-diamidino-2-phenylindole (DAPI);
tagging telomeres in the plurality of plasma cells through in situ hybridization with a telomere-specific labelled probe, optionally a peptide nucleic acid (PNA) probe,
mounting the test sample using an antifade mounting medium;
3D imaging the test sample; and
measuring on the 3D images values for the telomere parameters to obtain the 3D telomere organization sample signature.

In one embodiment, assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

In one embodiment, the 3D imaging comprises acquiring an image dataset of different planes of 3D q-FISH fluorescent signals and reconstructing a 3D image of the telomeres using deconvolution of the images performed with a constrained iterative algorithm, optionally using fluorescence microscopy and/or obtaining a stack of at least 50 images with a sample distance of 200 nm along a z direction and 102 nm in each of a x and a y direction.

In one embodiment, the 3D telomere organization sample signature is determined from interphase plasma cells.

In one embodiment, the telomere parameter (each or all) comprises an absolute value, a mean, a median, a ratio, a percentile, a quartile, a rank, a range (optionally a percentile range or an interquartile range), or a combination thereof.

In one embodiment, the sample is a diagnostic sample.

In one embodiment, the telomere parameters of the classification model are selected to have an accuracy of at least 70%, at least 75%, at least 80%, at least 85%, at least 90%, at least 95%, at least 96%, at least 97%, least 98%, at least 99% or 100% in distinguishing between stable SMM and high-risk SMM.

In one embodiment, the subject is a human subject.

Another aspect of the present disclosure is any method described herein, wherein when the subject is prognosed or diagnosed as having high-risk SMM, the subject is subsequently treated with one or more of lenalidomide, dexamethasone, siltuximab, daratumumab, bortezomib, elotuzumab, carfilzomib, thalidomide, cyclophosphamide and combinations thereof.

In one embodiment, the subject prognosed or diagnosed as having high-risk SMM is subsequently treated with: a) bortezomib and dexamethasone; b) siltuximab, c) daratumumab, lenalidomide, bortezomib and dexamethasone, d) elotuzumab, carfilzomib, lenalidomide, daratumumab, and dexamethasone, optionally for 3 to 4 cycles as induction therapy, e) bortezomib, lenalidomide and dexamethasone, f) bortezomib, thalidomide and dexamethasone, g) bortezomib, cyclophosphamide and dexamethasone, h) lenalidomide, or i) lenalidomide and dexamethasone.

In one embodiment is any method described herein, wherein when the subject is prognosed or diagnosed as having stable SMM or as having high-risk SMM, the subject is subsequently monitored.

In an embodiment, the method of monitoring a subject prognosed or diagnosed as having stable SMM or as having high-risk SMM comprises:
obtaining a subsequent sample from the subject, the subsequent sample comprising a plurality of plasma cells;
assaying the plurality of plasma cells according to any assaying step described herein, to obtain a 3D telomere organization monitoring signature;
applying a classification model to the 3D telomere organization monitoring signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates;
comparing the output classification of the 3D telomere organization monitoring signature to an output classification of a previous sample; and
providing an updated clinical outcome prognosis or an updated diagnosis.

In an embodiment, the method first comprises obtaining the test sample from the patient.

In an embodiment is a method of treating a subject with smoldering multiple myeloma (SMM) based on a 3D telomere organization signature from the subject, comprising administering to the subject a treatment selected from any treatment described herein when the subject has high-risk SMM, or monitoring the subject when the subject has stable SMM.

In an embodiment is a method of providing a personalized treatment plan for a subject with smoldering multiple myeloma (SMM) based on a 3D telomere organization sample signature of the subject, comprising providing the subject the personalized treatment plan to be administered to the subject when the subject has high-risk SMM or monitoring the subject when the subject has stable SMM determined according to a method provided herein, wherein the treatment plan comprises a treatment selected from any treatment described herein.

In an embodiment, the subject having high-risk SMM or the subject having stable SMM is prognosed or diagnosed as having high-risk SMM or stable SMM according to any method described herein.

Another aspect of the present disclosure is use of any method described herein for treating a subject with SMM.

In an embodiment is the prognosis or diagnosis determined using any method described herein for use in treating a subject with SMM.

In an embodiment, treating comprises administering to the subject a treatment selected from any treatment described herein when the subject has high-risk SMM, or monitoring the subject when the subject has stable SMM.

Another aspect of the present disclosure is an assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising the telomere parameters: a) nuclear telomere distribution, a/c ratio, and total telomere length; b) a/c ratio and telomere numbers; c) a/c ratio and nuclear telomere distribution, or d) a/c ratio and telomere aggregates; applying a classification model to the 3D telomere organization sample signature to obtain an output classification of stable SMM or high-risk SMM, the model being a model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; providing the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with stable SMM is not likely to progress to MM within 2 years; and selecting a therapy as described herein for the subject when the subject is identified as having high-risk SMM, or monitoring the subject when the subject is identified as having stable SMM.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 is a panel of representative FISH images showing myeloma plasma cells from 3 smoldering myeloma patients. The figure shows the positive staining of CD56 and CD138 specific for myeloma plasma cells in addition to the telomeres labeled using telomeres probe with Cy3 fluorochrome.

FIG. 2 is a representative three-dimensional images of 3D-telomere co-immuno-FISH conducted on SMM bone marrow biopsy sections. Cells were co-immuno-stained, then microscopy and image capture were conducted as described in the Methods section. Top left) representative 3D image from a short progression SMM patient showing telomeric aggregates (white arrow). Top right) representative 3D image from a long progression SMM patient. Bottom left) representative 3D image from a short progression SMM patient showing telomeric disc indicating cell cycle progression (white arrow). Bottom right) representative 3D image of SMM plasma cells showing differential staining including overlay of all channels; telomeres (Cy3), nuclear staining (DAPI); CD56 (Alexa 488) & CD138 (Alexa 647/Cy5).

FIG. 3 is a receiver operating characteristic (ROC) curve showing the ability of a classification model for predicting non-progressing vs progressing SMM. Area under the curve (AUC) is 0.8002. The points within the graph are labelled by observation number.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

### Definitions

The term “smoldering multiple myeloma” or SMM is asymptomatic precursor to multiple myeloma (MM) and is defined as a disease having i) serum monoclonal protein greater or equal to 3 g/dL, ii) clonal bone marrow plasma cells greater than 10% but less than 60% (however, if plasma cell infiltration is over 10% then 2 g/dL monoclonal protein is considered SMM not MGUS), and iii) no end-organ lesions (including hypercalcemia, renal insufficiency, anemia, and bone lesions). The term “stable SMM” or “SMM-stable” as used herein means SMM that is likely to remain stable for over 2 years (in other words not likely to progress to full stage MM for over 2 years) from time of diagnosis. The term “high-risk SMM” or “SMM-progression” as used herein means SMM that is likely to progress to full stage MM within 1 to 3 years from time of diagnosis, for example within 2 years from time of diagnosis. The assessment of “stable SMM” and “high-risk SMM” is performed based on telomeric organization of plasma cells from a subject sample. As shown herein, high-risk SMM patients can, for example, have increased telomere numbers, total and average telomere length (as demonstrated by their proxies—total and average telomere intensity), telomere aggregates, a/c ratio and/or nuclear volume, and/or changes in nuclear telomere distribution compared to stable SMM patients.

The term “multiple myeloma” or MM or “full stage MM” as used herein means a tumor or cancer composed of cells derived from the hematopoietic tissues of the bone marrow. Multiple myeloma is also known as plasma cell myeloma and is defined by the presence of serum monoclonal protein levels greater than or equal to 20 g/dL, clonal bone marrow plasma cells greater than or equal to 60%, and end organ damage (e.g., lytic bone lesions, anemia, hypercalcemia, renal failure). However, presence of end organ damage is sufficient to diagnose a patient with full stage MM regardless of monoclonal protein levels or clonal bone marrow plasma cell ratio.

The term “three-dimensional (3D) analysis” or “3D analysis” as used herein refers to any technique that allows the 3D visualization of cells, for example involving high resolution deconvolution microscopy, image restoration or deconvolution, visualization and image analysis. An example of 3D image analysis is provided in U.S. Pat. No. 7,801,682, issued Sep. 21, 2010 titled Method of Monitoring Genomic Instability Using 3D Microscopy and Analysis, herein incorporated by reference in its entirety.

The term “telomere numbers” as used herein means a sum of all telomeres and all telomere aggregates identified within a cell and/or group of cells in a test sample, for example a cell or sample from a subject.

The term “telomere aggregates” means telomeres found in close proximity that cannot be further resolved at fluorescence microscopy maximum optical resolution of 200 nm (Vermolen et al., 2005; Mai and Garini, 2006; Mai, 2010). Telomeric aggregates are not typically observed in normal cells.

The term “total telomere length” as used herein means a sum of the length of all telomeres within a given cell or group of cells in a test sample, for example a cell or sample from a subject. The term “average telomere length” as used herein means a mean telomere length, the mean calculated from all telomeres within a given cell or group of cells in a test sample, for example a cell or sample from a subject. Total telomere length and average telomere length are calculated from the relative fluorescent telomeric signal intensity of labelled telomeres, and thus “total telomere intensity” is a proxy for total telomere length and “average telomere intensity” is a proxy for average telomere length. Given that telomeres are regions of repetitive nucleotide sequences, the number of fluorescent probes targeting the repeat sequences that bind to a telomere is a function of its length. Consequently, the intensity of the resulting fluorescent signal is also a function of telomere length. The relative fluorescent telomeric signal intensity represents the length of the telomeres in fluorescent arbitrary units (f.a.u.), which can be subsequently converted into kilo base (kB) pair lengths. This conversion can be calculated through for example a conversion factor derived from cells such as Raji cells, a Burkitt's lymphoma cell line used as a control, as described in Mathur et al., 2014 [42], herein incorporated by reference in its entirety. Any cell line with a defined telomere length can be used in the determination of a conversion factor. As an example, once Raji cells (or other control cell) are harvested, half are used for telomere restriction fragment (TRF) analysis via Southern blot while the rest underwent PNA Q-FISH using identical conditions as set for plasma cells (e.g., using a 63×/1.4 oil plan apochromatic objective lens). Using TeloView™, an average telomere intensity is calculated for the Raji cells. This can be correlated with the telomere length resulting from the TRF analysis, deriving the conversion factor. The intensities can then be converted into base pair lengths using this conversion factor.

The term “a/c ratio” refers to a spatial distribution of telomeres within the nucleus that changes according to the phase of the cell cycle. This cell cycle-dependent distribution of telomeres can be described as an ellipsoid having two axes of equal or similar length (termed “a” and “b”), and one axis of a different length (termed “c”). The ratio between the length of the a-axis and c-axis describes the degree to which this distribution deviates from a sphere, i.e., where a/c=1 (Vermolen et al., 2005). The orientation of the telomere ellipsoid within a sample is not necessarily parallel or perpendicular to the microscope viewing/imaging plane (described by the x- and y-axis), especially in those cases where cells are analyzed within tissue sections.

The term “nuclear telomere distribution” or “three-dimensional (3D) distribution of telomeres in the nuclear space,” also called “3D distribution of telomeres in the nuclear space,” “nuclear telomere distribution”, “distribution of telomeres”, “distance from nuclear centre” in the art, refers to a telomere spatial distribution parameter distinct from a/c ratio that describes the 3D positioning of each telomere within the nuclear volume. 3D distribution of telomeres in the nuclear space can be an absolute value or a relative value. For example, the centre of a nucleus is identified by its x-, y-, and z-axis coordinates as oriented according to the microscope viewing/imaging plane. Each telomere or aggregate also identified by its x-, y-, and z-axis coordinates. For any one telomere, the distance is measured between the nuclear centre, and the telomere to obtain the absolute distance of the telomere from the nuclear center. The absolute distance of the telomere from the nuclear center can be averaged for each telomere measured within a cell to provide the average distance of the telomeres from the nuclear center for the cell.

The term “nuclear volume” as used herein means the volume of a cell nucleus. The nuclear volume can be calculated for example according to the 3D nuclear 4′,6-diamidino-2-phenylindole staining (DAPI) protocol described in Vermolen B J et al., (2005).

The term “telomeric organization”, “telomere organization” or “telomeres organization” as used herein refers to the 3D arrangement of the telomeres during any phase of a cell cycle and includes such parameters as 3D distribution of telomeres in the nuclear space, number of telomere aggregates per cell, number of telomeres per cell, telomere lengths (or intensities), a/c ratios, and/or nuclear volumes. “Telomere organization” also refers to the size and shape of the telomeric disk, captured for example in an a/c ratio and which is the organized structure formed when the telomeres condense and align during the late G2 phase of the cell cycle.

The term “telomere organization signature” as used herein refers to a set of values of telomere organization parameters of a cell, sample or subject, that can be used to classify the cell, sample or subject, for example as stable SMM or high-risk SMM. The criteria that define the differences include the telomere parameters (e.g. their values for): a) nuclear telomere distribution, a/c ratio, and total telomere length; b) a/c ratio and telomere numbers; c) a/c ratio and nuclear telomere distribution, or d) a/c ratio and telomere aggregates of a cell or a group of cells for example at least 5 cells, a least 10 cells, at least 15 cells, at least 20 cells, at least 25 cells or at least 30 cells of a cell sample. The telomere parameters can be averages, medians or a range such as an interquartile range (e.g. 25-75%) determined by ranking and dividing telomere values into quartiles obtained from each of the plurality of cells.

The term “3D telomere organization sample signature” as used herein refers to a telomere organization signature (e.g. set of values for telomere parameters) obtained from a cell or group of cells in a test sample, for example a cell or sample from a subject that is suspected of having SMM such as stable SMM or high-risk SMM.

The term “3D telomere organization reference signature” as used herein refers to a telomere organization signature corresponding to or derived from a group of samples and associated with a control population, disease population or disease severity and comprises values for a plurality of telomere organization parameters. For example, a reference telomere organization signature is optionally obtained from a plasma cell sample from a subject or group of subjects that is known as not having SMM, or as having stable or high-risk SMM.

The term “3D telomere organization monitoring signature” as used herein refers to a telomere organization signature obtained from a cell or a group of cells in a monitoring sample, for example a cell or sample from a subject being monitored for continued classification as stable SMM or for a transition to high-risk SMM.

The term “percentile” or “percentile telomere value” as used herein refers to a descriptive statistic of the data distribution for a telomere value. A percentile is a value below which a certain percentage of the data falls. For example, the 89^{th} percentile is the value below which 89% of the data is located and above which 11% of the data is located. A percentile telomere value can be used to set a threshold value for subject diagnosis or prognosis. A percentile range can also be used, for example by ranking the values and excluding the top x and bottom y percent, where x and y can be the same or different, for example excluding the top 10%, or top 15% etc. and the bottom 10% or bottom 15% etc. A percentile or percentile range telomere parameter value can be used as a telomere parameter within a classification model, for example an algebraic classification model, that discriminates between stable SMM and high-risk SMM.

The term “quartile” or “quartile telomere value” refers to a descriptive statistic of the data distribution for a telomere value that divides the total data into 4 parts. The first quartile (also called Q1) is the 25^{th} percentile, below which 25% of the data is present; the second quartile (also called Q2, or the median) is the 50^{th} percentile, below which 50% of data is present; the third quartile (also called Q3) is the 75^{th} percentile, below which 75% of the data is present; and the fourth quartile (also called Q4) is the 100^{th} percentile, below which 100% of the data is present. A quartile telomere value can be used to set a threshold value for subject diagnosis or prognosis. An inter-quartile range can also be used, for example by ranking the values and excluding the top 25% and/or the bottom 25%. A quartile or inter-quartile range telomere parameter value can also be used as a telomere parameter within a classification model, for example an algebraic classification model, that discriminates between, stable SMM and high-risk SMM.

The term “classification model” refers to a model that uses input, wherein the input is one or a plurality of values corresponding to the telomere parameters: a) nuclear telomere distribution, a/c ratio, and total telomere length; b) a/c ratio and telomere numbers; c) a/c ratio and nuclear telomere distribution, or d) a/c ratio and telomere aggregates, obtained from assaying a diagnostic sample, wherein the telomere parameters were found to be significantly different between patient groups, and wherein the model can classify the input into output categories, for example binary categories such as stable SMM and high-risk SMM. A classification model can, for example, be an algebraic classification model, for example a logistic regression model that can calculate the probability that an SMM patient is a high-risk SMM patient or stable SMM patient. The associated terms “to classify”, “classifying”, and “performing classification” refer to the operations performed by a classification model.

The term “sample” as used herein refers to any biological fluid (e.g., bone marrow or blood) comprising a plasma cell (e.g., plasma lineage cell) from a subject including a sample from a test subject, i.e. a test sample, such as from a subject with SMM, wherein the test sample comprises abnormal and/or cancer cells, and a control sample from a control subject, e.g., a subject without SMM. For example, the sample comprises a bone marrow sample or a liquid biopsy blood sample comprising plasma cells. The sample can comprise a liquid biopsy blood sample, for example a peripheral liquid biopsy blood sample, a fractionated liquid biopsy blood sample, a bone marrow sample, a biopsy, a frozen sample, a fresh sample, a cell sample, and/or a paraffin embedded sample or section thereof. For example, the sample is a diagnostic bone marrow biopsy sample.

As used herein, the term “cell” includes more than one cell or a plurality of cells or portions of cells. The sample may be from any animal, in particular from humans, and may be biological fluids (such as blood, serum, or bone marrow), tissue, or organ. The term “test cell” is for example a cell obtained or derived from a subject with SMM. In such an embodiment, the test cell includes, but is not limited to, a hematopoietic cancer cell or a cancer precursor cell. The term “control cell” is a suitable comparator cell e.g. a cell that is known of not having a hematopoietic cancer (e.g. negative control) or that is known as having a hematopoietic cancer or precursor syndrome (e.g. positive control).

The term “control” as used herein refers to a suitable comparator subject, sample, cell or cells such as non-SMM subject, or liquid biopsy blood sample, cell or cells from such a subject, for comparison to a subject, sample (e.g. test sample) cell or cells from a subject having SMM; or an untreated subject, cell or cells, for comparison to a treated subject, cell or cells, according to the context. “Control” can also refer to a value or reference signature representative of a control subject, cell and/or cells and/or a population of subjects etc.

The term “prognosis” as used herein refers to an expected course of clinical disease. The prognosis provides an indication of disease progression and includes for example, an indication of likelihood of recurrence, progression to cancer, e.g. MM, metastasis, death due to disease, tumor subtype or tumor type. The prognosis can comprise a good prognosis which corresponds to a good clinical outcome relative to the spectrum of possible clinical outcomes for the specific cancer or syndrome, and a poor prognosis, which corresponds to a poor clinical outcome relative to the spectrum of possible clinical outcomes for the specific cancer or syndrome. As used herein, “good prognosis” means a probable course of disease or disease outcome that has reduced morbidity and/or reduced mortality compared to the average for the disease or condition. As used herein, “poor prognosis” means a probable course of disease or disease outcome that has increased morbidity and/or increased mortality compared to the average for the disease or condition.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. For example, a subject identified as high-risk SMM (e.g. having a high-risk of progressing to MM) can be treated to prevent or delay progression to MM. Treatment methods comprise administering to a subject a therapeutically effective amount of a suitable therapeutic compound including for example therapeutic compounds described herein and optionally consists of a single administration, or alternatively comprises a series of applications. For example, the compounds described herein may be administered at least once a week. However, in another embodiment, the compounds may be administered to the subject from about one time per three weeks, or about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. In some instances, chronic administration may be required. For example, the compounds are administered to the subject in an amount and for a duration sufficient to treat the patient. “Treating” and “Treatment” can also mean monitoring progression of a subject without administering an amount of a compound such as one of the compounds disclosed herein. For example, where the subject is identified as having stable SMM (e.g. having low risk of progression to MM) the treatment can comprise actively monitoring or surveying the subject, for example by routinely (e.g. once a year or every other year) assessing one or more 3D telomere organization parameters in plasma cells of the subject. This allows the subject to receive beneficial care without having to receive unnecessary therapeutic compound or other such treatment.

As used herein, the phrase “effective amount” or “therapeutically effective amount” means an amount effective, at dosages and for periods of time necessary to achieve a desired result or predicted to achieve a desired result.

The term “subject” also referred to as “patient”, as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 25%, 0.1-20%, or 1-25%, 1-20%, 1%-15%, preferably 10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods

The present disclosure provides in various embodiments, methods for assessing and stratifying SMM patients as likely to remain stable or as high-risk and likely to progress to MM. The methods provided can also be used to provide appropriate treatments to SMM patients more likely to progress to MM.

Technology for three-dimensional (3D) organization of telomeres in cells such as abnormal or cancer cells has been developed and is disclosed in U.S. Pat. No. 7,801,682, issued Sep. 21, 2010, and U.S. Pat. No. 9,963,745, issued May 8, 2018, each of which are incorporated herein by reference in their entirety.

The inventors' research has shown that the 3D organization of telomeres is altered in cancer cells (Louis et al., 2005; Chuang et al., 2004; Mai and Garini, 2006). This basic finding led to an understanding of genetic changes in early cancer cells and proved that telomere organization is key to genome stability vs. instability (Mai and Garini, 2006; Mai and Garini, 2005; Louis et al., 2005): The inventors have demonstrated that each nucleus has a specific telomeric signature that defines it as normal or aberrant. Six criteria (also called “telomere organization parameters” herein) define this difference; 1) nuclear telomere distribution, 2) the presence/absence of telomere aggregate(s), 3) telomere numbers per cell, and 4) telomere sizes, 5) a/c ratios and 6) nuclear volumes.

As disclosed herein, 3D telomere analysis of diagnostic bone marrow samples is unexpectedly able to provide prognosis and diagnosis information in SMM subjects. For example, it is demonstrated that SMM patients can be classified as having stable SMM or as having high-risk SMM based on their telomere signature. For example, multivariate analysis of the telomere signature of an SMM patient can reliably classify the signature as indicative of stable SMM or high-risk SMM using telomere parameters, for example total telomere length, a/c ratio, nuclear volume, and nuclear telomere distribution.

Accordingly in an aspect, the disclosure includes a method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; and
optionally providing the clinical outcome prognosis or the diagnosis according to the output classification, the clinical outcome prognosis or the diagnosis being stable SMM or high-risk SMM, wherein the subject with high-risk SMM is likely to progress to multiple myeloma (MM) within 2 years and the subject stable SMM is not likely to progress to MM within 2 years.

The telomere parameters or one or more features thereof of the classification model was determined by logistic regression using a training dataset comprising 3D telomere organization signatures associated with stable SMM and 3D telomere organization signatures associated with high-risk SMM, which were obtained from SMM patients of known annotation as high-risk SMM or stable SMM. The comparing can be done, for example, multivariate analysis.

A classification model described herein may be derived from historical data from a pool of samples with known annotation as high-risk SMM or stable SMM. In an embodiment, the classification model is continually updated as further samples from patients with known annotation as high-risk SMM or stable SMM are collected and telomere parameters are measured and correlated.

Providing the clinical outcome prognosis or the diagnosis can be to the subject, or to a medical professional, hospital or other medical establishment, for example it can be satisfied by sending or transmitting the clinical outcome prognosis or diagnosis to a medical professional, hospital or other medical establishment that has a relationship with the subject. The medical professional, hospital or other medical establishment may then communicate with the subject. For example, the clinical outcome prognosis or diagnosis can be sent by email, mail, or fax or be available for retrieval electronically by any of the foregoing.

“A test sample from a subject having SMM” in any method disclosed herein is, for example, a sample from a subject that meets the clinical criteria for SMM, optionally known or unknown. The sample may for example be a diagnostic sample, wherein the subject and/or sample meet SMM criteria or may be obtained from a subject that has been diagnosed with SMM.

In an embodiment according to any method described herein, the test sample is a bone marrow sample. For example, the bone marrow sample is a diagnostic bone marrow biopsy sample. In another embodiment, the plasma cell is a peripheral liquid blood plasma cell that may be obtained through a blood sample, for example a liquid biopsy of peripheral blood. A plasma cell for example is characterized by abundant basophile cytoplasm, a prominent Golgi zone and an eccentrically located nucleus. In addition, they express the specific marker CD138 for immunohistochemical detection. Plasma cells can readily be identified using a light microscopic technique in bone marrow and blood smears. These cells can be isolated using methods known in the art.

In an embodiment according to any method described herein, the prognosis or the diagnosis is provided to the subject or the subject's medical professional, optionally at time of SMM diagnosis.

In an embodiment according to any method described herein, the test sample is a diagnostic biopsy. A diagnostic biopsy is taken for example prior to treatment. A diagnostic sample is a sample of tissue which defines the first time a disease is under investigation. For example, a bone marrow node biopsy can reveal a subject has monoclonal gammopathy of undetermined significance (MGUS), stable SMM, high-risk SMM or MM. The diagnostic biopsy can provide a basis to start a specific treatment.

In an embodiment according to any method described herein, the assaying comprises:
labelling nuclei of the plurality of plasma cells with a fluorescent nuclear stain or probe, for example wherein the fluorescent nuclear stain is 4′,6-diamidino-2-phenylindole (DAPI);
tagging telomeres in the plurality of plasma cells through in situ hybridization with a telomere-specific labelled probe, optionally a peptide nucleic acid (PNA) probe;
mounting the test sample using an antifade mounting medium;
3D imaging the test sample; and
measuring on the 3D images values for the telomere parameters to obtain the 3D telomere organization sample signature.

In an embodiment according to any method described herein, the telomere-specific labelled probe is a Cy3-labelled peptide nucleic acid (PNA) probe. In other embodiments, the labelled probe is a PNA probe linked to another fluorophore, for example Cy2, GFP, or Alexa Fluor 594.

Malignant plasma cells can be identified, distinguished, and/or isolated from other cells in a sample, for example by double-labeling the sample for peptides CD138 and CD56, given that malignant plasma cells can express both peptides CD138 and CD56.

Accordingly, in an embodiment according to any method described herein, the assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

In an embodiment according to any method described herein, the 3D imaging comprises acquiring an image dataset of different planes of 3D q-FISH fluorescent signals and reconstructing a 3D image of the telomeres using deconvolution of the images performed with a constrained iterative algorithm. The 3D imaging may be obtained according to the method described herein. For example, the 3D imaging comprises using fluorescence microscopy using a 63×/1.4 oil plan apochromatic objective lens. In an embodiment, the 3D imaging comprises obtaining a stack of at least 50 images with a sample distance of 200 nm along a z direction and 102 nm in each of a x and a y direction.

In an embodiment according to any method described herein, the 3D telomere organization sample signature is determined from interphase plasma cells.

Each of the telomere parameters of the classification model can, for example, be represented as an absolute value or as a descriptive statistic. Accordingly, in some embodiments according to any method described herein, the telomere parameters of the classification model (each or all) comprises an absolute value, a mean, a median, a ratio, a percentile, a quartile, a rank, a range (optionally a percentile range or an inter-quartile range), or a combination thereof. For example, a ratio can be a percentage. In an embodiment, the classification model comprises a rank. In an embodiment, the classification model comprises a quartile. In an embodiment, classification model comprises a range. In an embodiment, the range is an inter-quartile range, for example the range between Q1 and Q2, the range between Q1 and Q3, or the range between Q1 and Q4.

In an embodiment, the telomere parameter is a weighted value.

In an embodiment according to any method described herein, the sample is a diagnostic sample.

Several classification models are disclosed herein that can distinguish between stable SMM and high-risk SMM. In an embodiment, the telomere parameters of a classification model are selected to have an accuracy of at least 70%, at least 75%, at least 80%, at least 85%, at least 90%, at least 95%, at least 96%, at least 97%, least 98%, at least 99% or 100% in distinguishing between stable SMM and high-risk SMM. In an embodiment, accuracy is determined by the area under a receiver operating characteristic (ROC) curve.

In an embodiment, the 3D telomere organization sample signature and/or the one or more of the at least one telomere parameter of the classification model consists of the telomere parameters: nuclear telomere distribution, a/c ratio, and total telomere length.

In an embodiment, the 3D telomere organization sample signature and/or the one or more of the at least one telomere parameter of the classification model consists of the telomere parameters: a/c ratio and telomere numbers.

In an embodiment, the 3D telomere organization sample signature and/or the one or more of the at least one telomere parameter of the classification model consists of the telomere parameters: a/c ratio and nuclear telomere distribution.

In an embodiment, the 3D telomere organization sample signature and/or the one or more of the at least one telomere parameter of the classification model consists of the telomere parameters: a/c ratio and telomere aggregates.

In an embodiment, the subject is a human subject.

Approximately 10-15% of patients diagnosed with SMM will transition into full stage MM. The current practice for SMM patients is to monitor without treatment for the fact that the high-risk 10-15% of patients cannot be accurately identified. The methods herein described unexpectedly allow for early and accurate identification of subgroups of SMM subjects, namely subjects with high-risk SMM, who may benefit from receiving early treatment to delay or prevent transition to full stage MM.

Subjects identified as high-risk SMM may benefit from treatment modalities similar to those used for MM patients. MM patients can be treated for example with bortezomib, lenalidomide, dexamethasone (VRd). Alternative bortezomib-containing regimens include, for example, bortezomib-thalidomide-dexamethasone (VTd) or bortezomib-cyclophosphamide-dexamethasone (VCd), and can be used instead of VRd.

Accordingly, another aspect disclosed herein relates to a method of treating a subject with high-risk SMM, comprising identifying (e.g., through clinical outcome prognosis and/or diagnosis) the subject as having high-risk SMM or stable SMM; and if the subject is identified as likely to have high-risk SMM, administering to the subject a treatment herein disclosed, and if the subject is identified as likely to have stable SMM, monitoring the subject.

In an embodiment, the subject is identified (e.g., through clinical outcome prognosis and/or diagnosis) as having high-risk SMM or stable SMM according to any method disclosed herein.

In an embodiment, the subject identified as having high-risk SMM, optionally by any method described herein, is subsequently treated with an amount, for example a therapeutically effective amount, of one or more of lenalidomide, dexamethasone, siltuximab, daratumumab, bortezomib, elotuzumab, carfilzomib, thalidomide, cyclophosphamide and combinations thereof, for example wherein the therapeutically effective amount with respect to a combination means an amount of each compound of the combination that is therapeutically effective when combined with the other compounds of the combination.

In one embodiment, the subject prognosed or diagnosed as having high-risk SMM is subsequently treated with: a) bortezomib and dexamethasone; b) siltuximab, c) daratumumab, lenalidomide, bortezomib and dexamethasone, d) elotuzumab, carfilzomib, lenalidomide, daratumumab, and dexamethasone, optionally for 3 to 4 cycles as induction therapy, e) bortezomib, lenalidomide and dexamethasone, f) bortezomib, thalidomide and dexamethasone, g) bortezomib, cyclophosphamide and dexamethasone, h) lenalidomide, or i) lenalidomide and dexamethasone.

For example, the treatment is or comprises bortezomib and dexamethasone.

For example, the treatment is or comprises siltuximab.

For example, the treatment is or comprises daratumumab, lenalidomide, bortezomib and dexamethasone.

For example, the treatment is or comprises elotuzumab.

For example, the treatment is or comprises carfilzomib, lenalidomide, daratumumab, and dexamethasone, optionally for 3 to 4 cycles as induction therapy.

For example, the treatment is or comprises bortezomib, lenalidomide and dexamethasone.

For example, the treatment is or comprises bortezomib, thalidomide and dexamethasone.

For example, the treatment is or comprises bortezomib, cyclophosphamide and dexamethasone.

For example, the treatment is or comprises lenalidomide.

For example, the treatment is lenalidomide and dexamethasone.

A subject identified as having stable SMM may benefit from monitoring to determine whether the subject has remained in stable SMM or whether the subject has transitioned to high-risk SMM when they may benefit from treatment modalities for MM patients. A subject identified as having high-risk SMM may also benefit from monitoring, for example monitoring the response to any of the treatments disclosed herein. The monitoring can comprise comparing the output classification of a 3D telomere organization monitoring signature to an output classification of a previous sample from the subject. Alternatively, the monitoring can comprise comparing a probability that a subject has high-risk SMM as derived from a subsequent sample to the previous probability that the subject had high-risk SMM as derived from the previous sample. For example, the monitoring can comprise providing an updated probability, or a change in probability, that the subject has high-risk SMM or stable SMM. Reference to “the subject has high-risk SMM” refers to identification of the patient as having high-risk SMM and reference to “the subject has stable SMM” refers to identification of the patient as having stable SMM. Accordingly, “the subject has high-risk SMM” and “the subject is identified as having high-risk SMM” can be used interchangeably and similarly “the subject has stable SMM” and “the subject is identified as having stable SMM” can be used interchangeably.

Accordingly, in an embodiment, the subject prognosed or diagnosed as having stable SMM or as having high-risk SMM is subsequently monitored.

In an embodiment, the method of monitoring a subject prognosed or diagnosed as having stable SMM or as having high-risk SMM comprises:
obtaining a subsequent sample from the subject, the subsequent sample comprising a plurality of plasma cells;
assaying the plurality of plasma cells according to any assaying step described herein, to obtain a 3D telomere organization monitoring signature;
applying a classification model to the 3D telomere organization monitoring signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates;
comparing the output classification of the 3D telomere organization monitoring signature to an output classification of a previous sample; and
providing an updated clinical outcome prognosis or an updated diagnosis.

For example, the subsequent sample can be obtained, for example, 6 months, 1 year, 2 years, 3 years, 4 years, 5 years, or 10 years following prognosis or diagnosis as having stable SMM or as having high-risk SMM, optionally wherein a new subsequent sample from the subject is obtained, for example, every 6 months, every year or every other year.

In an embodiment, any method described herein first comprises obtaining the test sample from the patient.

Another embodiment herein disclosed is a method of treating a subject with smoldering multiple myeloma (SMM) based on a 3D telomere organization signature from the subject or a classification model, comprising administering to the subject a treatment, for example a treatment selected from the treatments disclosed herein when the subject has high-risk SMM, or monitoring the subject when the subject has stable SMM.

In an embodiment, the method of treating a subject with smoldering multiple myeloma (SMM) based on a 3D telomere organization signature from the subject or a classification model is performed on a subject that has been prognosed or diagnosed as having high-risk SMM or stable SMM according to any method provided herein.

Another embodiment herein disclosed is a method of providing a personalized treatment plan for a subject with smoldering multiple myeloma (SMM) based on a 3D telomere organization sample signature of the subject or a classification model, comprising providing the subject a treatment plan to be administered to the subject when the subject high-risk SMM and monitoring the subject when the subject has stable SMM.

In an embodiment, the method of providing a personalized treatment plan for a subject with smoldering multiple myeloma (SMM) based on a 3D telomere organization signature of the subject is performed on a subject that has been prognosed or diagnosed as having high-risk SMM or stable SMM according to any method provided herein.

Another embodiment herein disclosed is use of a method described herein for treating a subject with smoldering multiple myeloma (SMM), for example, wherein treating comprises administering to the subject a treatment described herein when the subject has high-risk SMM, or monitoring the subject when the subject has stable SMM.

Yet other embodiments herein disclosed are uses of the prognosis determined using a method described herein in treating a subject with smoldering multiple myeloma (SMM), for example, wherein treating comprises administering to the subject a treatment such as a treatment described herein when the subject has high-risk SMM, or monitoring the subject when the subject has stable SMM.

Another aspect herein disclosed is an assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates; applying a classification model to the 3D telomere organization sample signature to obtain an output classification of stable SMM or high-risk SMM, the model being a model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates, for example an algebraic model; providing the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with of stable SMM is not likely to progress to MM within 2 years; and selecting a therapy according any one herein disclosed for the subject when the subject is identified as having high-risk SMM, or monitoring the subject when the subject is identified as having stable SMM.

In an embodiment, the telomere parameter is determined by dividing telomere values into quartiles, as described herein.

In an embodiment, the 3D telomeric organization signature is determined using 3D quantitative fluorescence in situ hybridization (3D q-FISH).

In an embodiment, 3D q-FISH comprises labelling the telomeres with a telomere FISH protocol as described in the Examples. For example, the telomere FISH protocol can be performed by using Cy3-labelled peptide nucleic acid (PNA) probes (DAKO). In an embodiment, telomere labeling is done through in situ hybridization. In an embodiment, the nucleus is labeled with a fluorescent nuclear stain or probe, for example wherein the fluorescent nuclear stain is 4′,6-diamidino-2-phenylindole (DAPI). In an embodiment, 3D q-FISH comprises labelling CD138 in the plurality of plasma cells with a CD138-specific antibody linked to a fluorescent label and/or labelling peptide CD56 in the plurality of plasma cells with a CD56-specific antibody linked to a fluorescent label prior to the mounting of the test sample. In an embodiment, the sample is mounted, optionally using an antifade mounting medium.

For example, 3D Image Acquisition and Analysis can be performed as described in the Examples. For example, the 3D images can be obtained using a 3D imaging system that enables an Abbe resolution of 200 nm, for example an AxioImager Z1 (Zeiss) microscope using a 63×/1.4 oil plan apochromatic objective lens. In an embodiment, imaging is done on interphase plasma cells. In an embodiment, 3D imaging of telomeres is performed by acquiring a stack of up to 80 images along the z-axis (specimen depth), with a z-axis step of 0.2 μm between each image in the stack. In an embodiment, The sampling distance in both the x- and y-planes is 102 nm. In an embodiment, the exposure time for Cy3 (telomeres) is maintained at 100 milliseconds. In an embodiment, images are acquired in multichannel mode. In an embodiment, the images are deconvolved using a constrained iterative algorithm. In an embodiment, the method uses Teloview™. For example, Teloview™ can be used to determine the 3D telomere organization of a cell. TeloView™ is capable of scanning multiple cells, multiple cell types and multiple patients at one time. In an embodiment, the 3D Image Acquisition can involve analyzing plasma cells with deconvolution module and rendering module. In an embodiment, the 3D image can consist of a stack of at least 51 images with a sampling distance of 200 nm along the z direction and 102 nm in the x and y direction for every fluorochrome.

In an embodiment, the 3D Image Analysis for telomeres involves telomere measurements done with TeloView™. In an embodiment each telomere or telomere aggregate is given a set of coordinates (x, y, z) denoted by crosses on the screen in TeloView™. In an embodiment, total telomere length, average telomere length, telomere numbers, telomere aggregates, a/c ratio, nuclear volume, nuclear telomere distribution, or a combination thereof is measured using TeloView™.

In an embodiment, statistical analysis is performed on measures derived from total telomere length, average telomere length, telomere numbers, telomere aggregates, a/c ratio, nuclear volume, nuclear telomere distribution, or a combination thereof. In an embodiment, statistical analysis is used to identify differences and similarities between a stable SMM 3D telomere organization reference signature and a high-risk SMM 3D telomere organization reference signature, the stable SMM 3D telomere organization reference signature defining for one or more of the parameters of a 3D telomere organization signature associated with stable SMM and the high-risk SMM 3D telomere organization reference signature defining for one or more of the parameters a 3D telomere organization signature associated with high-risk SMM. In an embodiment, bivariate analysis of the stable SMM 3D telomere organization reference signature and the high-risk SMM 3D telomere organization reference signature is used to identify a list of potential telomere parameters or features thereof to use in a classification model, optionally an algebraic model. In another embodiment, statistical analysis comprises multivariate analysis. In an embodiment, multivariate analysis comprises using forward selection methodologies, backward selection methodologies or a combination thereof. In an embodiment, multivariate analysis identifies one or more telomere parameters or features to use in a classification model, optionally an algebraic model. In an embodiment, the accuracy of the classification model is determined by the area under the receiver operating characteristic (ROC) curve. In an embodiment, the telomere parameters or features thereof of the classification model comprises an absolute value, a mean, a median, a ratio, a percentile, a quartile, a rank, and a range (optionally a percentile range or an inter-quartile range), or a combination thereof. For example, a ratio can be a percentage. In an embodiment, the classification model comprises a rank. In an embodiment, the classification model comprises a quartile. In an embodiment, classification model comprises a range. In an embodiment, the range is an inter-quartile range, for example the range between Q1 and Q2, the range between Q1 and Q3, or the range between Q1 and Q4. In an embodiment, the classification model is applied to the 3D telomere organization sample signature of a subject having SMM to obtain an output classification of stable SMM or high-risk SMM.

In an embodiment, the classification model can comprise a rank of one or more of the telomere parameters. For example, one or more of the telomere parameters can be ranked from lowest to highest. In another embodiment, the classification model can comprise calculating an inter quartile range for one or more of telomere parameters, for example the 75^{th} to 25^{th} percentile range. In another embodiment, the interquartile range is calculated for at least one ranked telomere parameter. In one embodiment, the two or more telomere parameters are a/c ratio, telomere distribution and number of telomeres. In an embodiment, one or more of the telomere parameters is weighted.

In one embodiment, the cells for each patient are analyzed and the telomere parameters are measured. Some telomere parameters, such as a/c ratio and total telomere length, produce a single value per cell. Other telomere parameters, such as nuclear telomere distribution, produce one value for each telomere or telomere aggregate within a cell.

In one embodiment, the telomere parameters that produce multiple values per cell, optionally nuclear telomere distribution, are averaged for each cell such that each cell analyzed has one value per telomere parameter.

In one embodiment, for each of the telomere parameters of a patient, the values determined for each cell are ranked from lowest to highest. For example, a telomere parameter for each cell is determined such as average distance to the nucleus. If 20 cells are assessed, the telomere parameters determined for each of the 20 cells is ranked. In another embodiment, the value representing or encompassing the 25^{th} percentile and the 75^{th} percentile of the ranked values are selected. In yet another embodiment, for each of the telomere parameters of a patient, the value representing or encompassing the 25^{th} percentile is subtracted from the value representing or encompassing the 75^{th} percentile, to produce an inter-quartile range. The interquartile range for example of 20 cells would for example be the value for the 5^{th} highest value less the value for the 15^{th} highest value.

In an embodiment, the interquartile range for each of the telomere parameters is used as a variable in multivariate logistic regression analyses for calculating probability of high-risk SMM. In another embodiment, the multivariate logistic regression analyses are used to determine the weighting to be applied to each variable in order to optimize the resultant receiver operating characteristic (ROC) area-under-the-curve (AUC).

In an embodiment, a threshold value is derived from the ROC curve. In another embodiment, a patient with a probability of high-risk SMM above the threshold value is classified as a high-risk SMM patient and/or a patient with a probability below or equal to the threshold value is classified as a stable SMM patient.

The classification models as described herein can predict the probability that a subject has high risk SMM. A threshold probability can be selected such that when the probability is above the selected threshold, the subject is identified as having high-risk SMM and when below the selected threshold the subject is identified as having stable SMM.

The threshold value can be calculated, for example based on a patient population as described herein. In yet another embodiment, the threshold value is 0.254. Depending on the selectivity and sensitivity desired, other threshold values can be used. When a patient has a probability that is above the threshold value, the patient is likely to progress (i.e. have high-risk SMM). If the patient has a probability that is below the threshold, the patient is unlikely to progress.

Also provided is a computer implemented method of clinical outcome prognosis of a subject with SMM.

In an embodiment, the method comprises: at a computer system having one or more processors, and memory storing one or more programs for execution by the one or more processors:
a) obtaining, in electronic format, a 3D telomere organization sample signature of a test sample from the subject, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates; and
b) applying a classification model to the 3D telomere organization sample signature to obtain an output classification of stable SMM or high-risk SMM, the model being a model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates;
wherein the classification model provides an indication of the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with stable SMM is not likely to progress to MM within 2 years.

In an embodiment, the test sample is a subsequent sample and the method is used for monitoring the subject with SMM, optionally stable or high-risk SMM. The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1—Predicting Disease Progression of Individual Smoldering Myeloma Patients with High Accuracy (Training Cohort)

### Materials and Methods

### Study Design and Patient Information

All patients were consented in accordance with the Declaration of Helsinki (fifth revision from 2000 with Clarifications of Articles 29, 30 (2002-2004). The study included a total of 168 patients diagnosed with SMM according to standard International Myeloma Working Group (IMWG) guidelines approved in 2014. Two independent cohorts comprised the total number of patients in the study. One cohort included 92 patients and was used as a training dataset (cohort 1, Example 1) to develop a predictive scoring model, and a second cohort of 76 patients was used for a blinded validation (cohort 2, Example 3). Patients were selected for the study based on previously established inclusion and exclusion criteria (Table 1) including short progression SMM patients, who progressed to symptomatic MM stage within 24 months, and long progression SMM patients who remained stable for >5 years.

### Tissue Specimens

The study was conducted using archived sections of the initial diagnostic bone marrow biopsy collected from each patient according to standard tissue collection and processing procedures. The biopsies were paraffin embedded according to the guidelines of the College of American Pathologists (CAP). The collected tissue was B-5 fixed at time of collection and decalcified according to standard procedures. Sections of 5 μm thickness were used for the quantitative 3D-Telomere co-Immuno-FISH procedure.

### Quantitative 3D Telomere Co-Immuno-FISH Assay

The tissue section placed on a glass slide was deparaffinized using two changes of Xylene, immersed into two changes of 100% Ethanol and two changes of 95% Ethanol for 3 minutes each. To remove the mercury deposits from the tissue (typical to B-5 mercury-based fixative) a pigment removal treatment using Lugol solution for 20 minutes followed by a treatment with sodium thiosulfate for 3 minutes was employed. The pigment removal was followed by Antigen Retrieval (S169984-2, Agilent, Canada) treatment for 30 minutes at 95° C. For quantitative 3D-telomere FISH hybridization a Cy-3 labeled PNA telomere probe was applied (Agilent, USA). Denaturation was performed at 80° C. for 3 minutes followed by hybridization at 30° C. for two hours. The denaturation and hybridization were performed using a ThermoBright machine (Leica Biosystems, USA). Following hybridization, the sides were washed twice in washing buffer I (70% Formamide/10 mM Tris pH 7.5±1) at room temperature for 15 minutes each and then in a washing buffer II (0.1× saline-sodium citrate (SSC, pH: 7.5±1)) for 5 minutes at 55° C. To proceed with the immunostaining component of the co-immuno-F/SH, slides were blocked in Super Block buffer (Sigma) for 5 minutes at room temperature. A cocktail of Mouse Anti-Human CD56 antibody directly labelled with Alexa Fluor®488 (BD Bioscience, San Jose, CA, USA) and anti-human CD138 (Syndecan-1) (Biolegend, San Diego, CA, USA) directly labelled Alexa Fluor®647 was applied at a dilution of 1:50 for each antibody. After incubation, slides were washed to remove any excess unbound antibodies, then the nuclei were counterstained with 4′6 diamidino-2-phenylindole (DAPI) for visualization followed by application of antifade mounting medium Vectashield (Vector Laboratories, Burlington, Ontario).

### 3D Image Acquisition & Processing

Images were acquired using a Zeiss AxioImager Z.2 microscope (Zeiss, Jena, Germany) equipped with a Hamamatsu digital camera (C11440-42U). Using an artificial intelligence algorithm, the slides were first scanned with a 10× objective to identify areas on the slides that were rich with target cells based on the positive co-immuno staining with the anti-CD56 and anti-CD138. 3D images were then acquired using an automated batch imaging program using a 63× Zeiss immersion objective. Fifty-one focal planes spaced at 200 nm were captured along the Z-axis (specimen depth) for every fluorochrome including Cy3 filter (telomeres) at 150 msec, Spectrum green (CD56) at 400 msec, Cy5 (CD138) at 400 msec and DAPI (DNA) at 20-40 msec. Except for DAPI counterstain, the exposure time was kept constant throughout the entire study. The exposure for DAPI was assessed on a slide-by-slide basis. Images of individually selected cells were then processed using the constrained iterative deconvolution algorithm (Zeiss) to reconstruct a 3D image using the acquired 51 focal planes.

### TeloView® Analysis

Telomere parameters were quantified in 3D using the TeloView® software platform (TeloViewNet v1.18) proprietary to Telo Genomics, Toronto, Canada (Vermolen et al 2005 Cytometry). TeloView® quantifies 6 primary molecular and structural telomeric parameters including: 1—telomere length as a function of signal intensity; 2—number of telomere signals/nucleus; 3—number of telomeric aggregates (clusters of telomeres that are too close to be further resolved at an optical resolution limit of 200 nm); 4—nuclear volume; 5—a/c ratio (spatial feature that assesses cell cycle progression and is a measure of proliferation and progression); and 6—the distribution of telomeres within the nuclear space (spatial feature that informs on gene expression). Other relevant parameters may be derived from the 6 primary measured parameters including clustering telomeres based on size, average and total telomere length and the average and total number of telomere signals/nucleus, the percentage of telomeres stumps (i.e., very short telomeres), and the percentiles or quartiles of the individual parameter quantification.

### Statistical Analysis

General univariate t-test procedures were used to assess the relationship between the measured telomere parameters of the patient groups and to identify the parameters that were significantly different between the 2 groups and could be used as predictors in regression modeling. First, the telomeric parameters were assessed for equality of variances using Levene's test. For the parameters with equal variance, a student's t-test was performed; for the parameters with unequal variance a Welch's t-test was performed.

The identified predictors were then verified for their suitability for modeling using confidence interval representation and NAPR1WAY analysis of variance. Multivariate logistic regression analysis was employed to generate predictive models using the identified suitable predictors. Receiver Operating Curve (ROC) curves were generated for the developed predictive models to assess their predictive power and to plot the corresponding sensitivity and specificity. The generated predictive models were assessed for confidence using the Likelihood Ratio, Wald, and Score tests.

### Cohort Clinical Data and Outcome

The studies of Example 1 and Example 3 together included a total of 168 SMM patients. All patient biological samples and clinical data were provided by The Mayo Clinic and were processed in Telo Genomics Central Laboratory, Toronto, Canada, based on a data sharing agreement. All patients included in the cohort were initially diagnosed with SMM based on the standard criteria approved by the IMWG guidelines in 2014 [44]. The study inclusion and exclusion criteria are listed in Table 1. Of the 168 patients, the samples of 162 patients were processed successfully while 6 samples failed either due to compromised tissue quality or technical issues encountered during processing, with an overall study inclusion rate of 96%. The 168 patients were divided into 2 independent cohorts. The first cohort, i.e., the cohort of Example 1, included 92 patients (88 patients processed successfully) including 32 patients who progressed to symptomatic MM within 24 months (i.e., short progression) and 56 patients who remained in the SMM stage for 5+ years (i.e., long progression). This cohort was used as a training dataset to identify significant predictors and develop a predictive classification model. The second cohort, i.e., the cohort of Example 3, included 74 patients (72 patients processed successfully) including 45 patients with short progression and 29 patients with long progression intervals.

TABLE 1
List of study inclusion and exclusion criteria

| Inclusion Criteria | Exclusion Criteria |
|---|---|
| Diagnosis of SMM by IMWG 2014 criteria | Enrolled on a treatment trial |
| 2 subsets of patients: | Progression to MM within >2 < 5 years |
| a) Progression to MM within 2 years of SMM | |
| diagnosis | |
| b) Progression to MM beyond 5 years from | |
| SMM diagnosis | |
| Complete follow up data to ensure progression | Incomplete diagnosis criteria |
| Availability of clinical diagnosis criteria including: | |
| serum free light chain ratio; % of MM plasma cells | |
| & M-protein level at time of diagnosis | |

### Target Myeloma Plasma Cells Identification and Confirmation

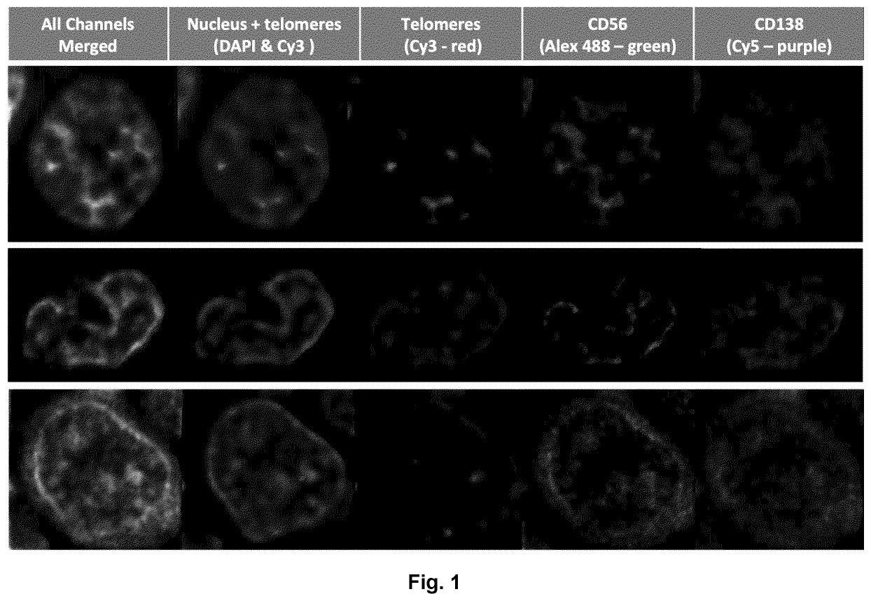

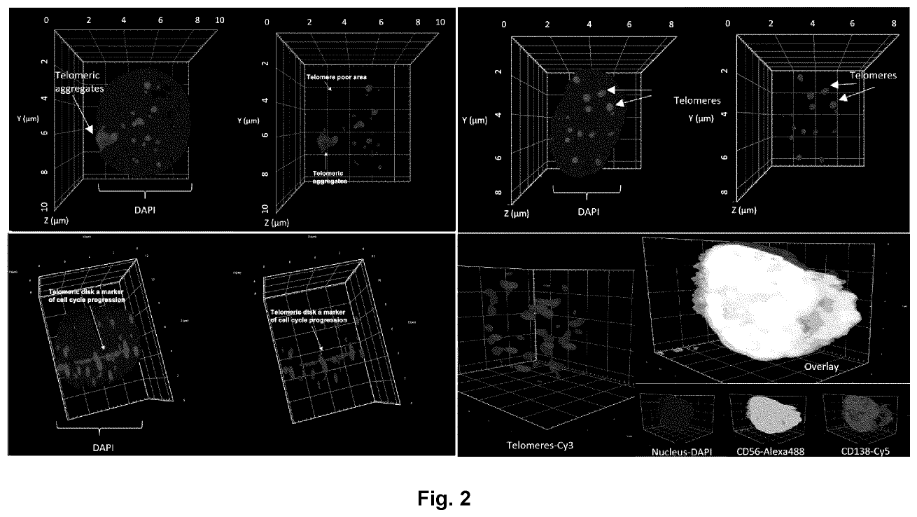

Single MM cells were selected from the captured microscopy fields based on positive co-immunostaining with anti-CD56 and anti-CD138 antibodies specific to MM plasma cells (FIG. 1 and FIG. 2). Cells were first selected using Telo Genomics proprietary automated cell selection tool and were then reviewed and approved by a trained technologist. Only cells confirmed by a trained technologist were considered for the analysis. Fifty positive MM plasma cells were analyzed for each patient using the TeloView software.

### Univariate Statistics Analysis and Identifications of Significant Predictors:

Initial univariate and bivariate analyses using simple nested methodologies including Anova, Supervised and Unsupervised student t-test were conducted to identify telomeric parameters that were significantly different between the short progression and long progression patient groups. The univariate tests were applied to the mean of quantification of each parameter across the 2 patient groups, and statistical significance was considered if the p-value was <0.05 (Table 2). The analysis identified 4 significant independent and derived telomere parameters including: 1—total telomere length, 2—a/c ratio, 3—nuclear telomere distribution, and 4—the nuclear volume (see Table 2). These parameters were considered predictors suitable for regression modeling. A sample of the data from 10 patients, selected at random, is provided in Table 6, below.

TABLE 2
The significant progression predictors and their respective p-values

| | | Equality of |
|---|---|---|
| Telomere Parameter | p-value | variance |
| Nuclear telomere distribution | 0.02 | <0.0001 |
| a/c ratio | 0.001 | <0.0001 |
| Nuclear volume | 0.03 | <0.001 |
| Total telomere length | 0.03 | <0.0001 |

### Results

### Multivariate Modeling

Predictive modeling was performed using ROC curve analysis including different combinations of all the suitable predictors and their derivatives to determine which combination of predictors would yield the highest Area Under the Curve (AUC), specificity and sensitivity, using the data of the 88 patients as a training dataset. From the bivariate analysis of the two groups (i.e., stable SMM and high-risk SMM), a list of predictors was compiled. These predictors were found to discriminate between the two groups generally, using a significance level of p<0.30. As these predictors may come from the same parameter, though aggregated differently (e.g., 25th percentile, 50th percentile, 75th percentile, mean, and inter-quartile ranges—distance from 25th percentile to 75th percentile) from the cells studied, such highly correlated predictors were avoided by including them separately in the stepwise analysis.

Two types of stepping procedures were used to analyze the potential predictors—forward stepwise and backward stepwise, additionally predictive predictors were added forward or deleted backward with level set at p=0.06 to select and p=0.05 to stay. The models found to meet fit Akaike information criterion (AIC) as well as global likelihood ratio tests were selected, and their receiver operating characteristic (ROC) curves (sensitivity and specificity points) plotted. An estimate was made for each predictor in the beta coefficients, odds ratios along with their confidence intervals. These coefficients were adjusted for the other predictors in the model. The output of the ROC was used to select the sensitivity and specificity that best serve clinical utility. Several predictive classification models were achieved (Table 3).

TABLE 3
Area-under-the-curve (AUC) of predictive models

| | Corresponding |
|---|---|
| Predictors | AUC |
| 3 predictors: nuclear telomere distribution, a/c ratio & total | 0.80 |
| telomere length | |
| 2 predictors: a/c ratio & telomere numbers | 0.75 |
| 1 predictor: a/c ratio only | 0.64 |
| 2 predictors: a/c ratio & total number of telomere aggregates | 0.77 |
| 2 predictors: a/c ratio & nuclear telomere distribution | 0.78 |

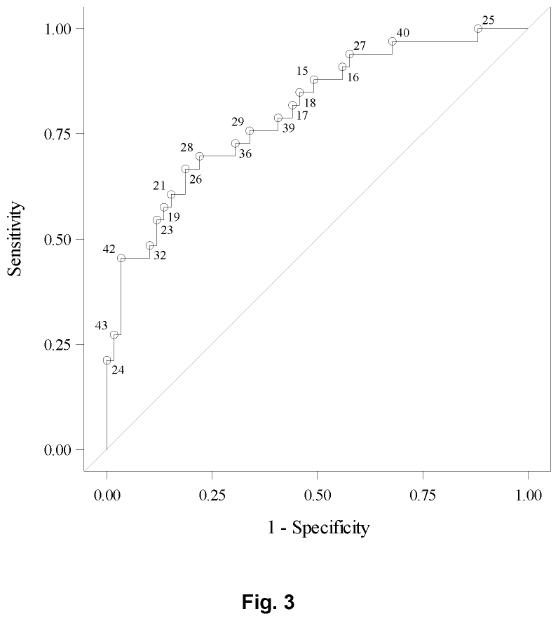

Of the generated models, the highest AUC, specificity, and sensitivity were achieved using 3 predictors including the total telomeres length, a/c ratio and nuclear telomere distribution. The generated ROC curve had an AUC of 0.8 (i.e., accuracy of 80%), with plotted specificity and sensitivity of 0.75 and 0.70, respectively (FIG. 3). The number provided for each data-point in FIG. 3 represents the number of patients in the training cohort that corresponded to that data point. The confidence of the generated model was examined using the Likelihood ratio, Wald and Scoring tests. The model showed high confidence with p-values of <0.001 in all 3 tests (Table 4).

TABLE 4
Confidence of predictive classification model as assessed
by Likelihood Ratio, Scoring & Wald tests.

| Confidence test | Significance |
|---|---|
| Likelihood Ratio | 0.0003 |
| Scoring | 0.0009 |
| Wald | 0.0024 |

In contrast to the above, neither telomere numbers nor nuclear telomere distribution was found to be significant for SMM comparisons (i.e., SMM vs MM, SMM stable vs SMM high progression) in a previously reported study [43]. The combinations in Table 2 result in models with improved predictive ability over those previously reported.

The results of this study present an independent prognostic biomarker able to stratify SMM patients into their respective risk groups, allowing for evidence-based treatment decision of high risk SMM patients while minimizing unnecessary treatment in stable SMM patients. Equally important, these results present a means to confidently monitor low risk SMM patients to confirm the stability of the SMM disease over time.

### Example 2: Model for Risk of Disease Progression of Individual Smoldering Myeloma Patients

The predictors 1) nuclear telomere distribution, 2) a/c ratio, and 3) total telomere length (Table 2, top row) were used to develop a logistic regression model that determined the probability that an SMM patient was a high risk SMM patient using the methods identified in Example 1. This logistic regression model comprised the following methods and calculations.

As described in Example 1, 50 cells for each patient were analyzed and telomere parameters were measured. Some telomere parameters, such as a/c ratio and total telomere length, produce a single value per cell. Other telomere parameters, such as nuclear telomere distribution, produce one value for a telomere or telomere aggregate within a cell. Telomere parameters that produced multiple values per cell were averaged for each cell, such that each cell analyzed had one value per telomere parameter.

For each of the three predictors, and for each patient, the values of the 50 cells were ranked from lowest to highest. The value representing the 25^{th} percentile and the 75^{th} percentile of the ranked values was selected.

For each of the three predictors and for each patient, the value representing the 25^{th} percentile was subtracted from the value representing the 75^{th} percentile to produce an inter-quartile range. Inter-quartile range was a suitable and representative statistic to derive from these datasets, as the distribution of the data was skewed rather than being normally distributed. This statistic accounted for 50% of the data, while limiting the impact of outliers at either end of the distribution.

The interquartile range for each of nuclear telomere distribution, a/c ratio, and total telomere length were used as the three variables in multivariate logistic regression analyses to determine the weighting to be applied to each variable in order to optimize the resultant receiver operating characteristic (ROC) area-under-the-curve (AUC).

From the ROC curve produced from the optimized equation, the numerical threshold 0.254 was derived, wherein a patient with a probability of progressing to MM above 0.254 was deemed to be a high-risk SMM patient and a patient with a probability below or equal to 0.254 was deemed to be a stable SMM patient. This threshold was selected to maintain high sensitivity while limiting the number of false negatives.

To predict the probability that any one SMM patient was a high risk SMM patient using this model, the interquartile range of the three predictors was calculated for the patient as above and applied to the optimized equation. The resultant probability was compared to the threshold 0.254, and the patient was classified as a high-risk SMM patient or a stable SMM patient, as appropriate.

### Example 3: Validating Model of Disease Progression of Individual Smoldering Myeloma Patients (Validation Cohort)

The second cohort of patients identified in the methods of Example 1 was used blindly to validate the best classification model identified in Example 1 and described in Example 2, and to calculate the assay performance characteristics including positive predictive value (PPV), negative predictive value (NPV), specificity and sensitivity. The inventors were blinded to patient outcome until all the samples were collected and all the raw data was generated. The statistics analysis was conducted by an independent statistician.

The samples of the second cohort with 74 SMM patients were received after the completion of the processing and analysis of the initial cohort used as a training dataset. Of the 74 patients, samples from 72 were processed successfully. This cohort included 45 short progression patients and 29 long progression patients. The inventors were blinded to the patient outcome. The raw data from these patients was examined by the predictive classification model developed using the training dataset (Examples 1 and 2). Probability and predicted outcomes for each patient were generated and the predicted outcome was then cross matched with the patient's clinical outcome. The classification model correctly identified 36 out of the 45 short progression patients (9 false negative). Similarly, the classification model correctly identified 22 out of the 29 long progression (7 false positive). Based on the true/false positive and negative scores, the blind validation revealed PPV of 84% and a NPV of 71% (Table 5), respectively, with a corresponding sensitivity and specificity of 80% and 76%, respectively.

TABLE 5
Blind validation matching outcome

| | Total number | True | True | |
|---|---|---|---|---|
| | of cases | Positive | Negative | PPV/NPV |
| High-Risk SMM | 45 | 39 (9 false | N/A | PPV: 84% |
| | | negative) | | |
| Stable SMM | 29 | N/A | 22 (7 false | NPV: 71% |
| | | | positive) | |

The blind validation using the optimized model in Example 2 with the threshold 0.254, revealed 36 true positive, 9 false negative, 22 true negative & 7 false positive (Table 3). The test characteristic based on the blind validation include: PPV of 84%, NPV of 71%, sensitivity of 0.8 and specificity of 0.76.

This model again performed classification with high sensitivity and specificity in a separate cohort of patients with SMM. This further indicates the predictive power of the telomere parameters assessed.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

TABLE 6
Subset of data from 10 random patients in the first cohort (training cohort)

| | Total | | | Total | Nuclear |
|---|---|---|---|---|---|
| | no. of | Total no of | | telomere | Telomere |
| Case | telo. | aggregates | AC Ratio | length | Distribution |
| Patient 1 | 19 | 3 | 1.9101 | 831196 | 2726.38 |
| Patient 1 | 13 | 1 | 1.5560 | 369378 | 1521.57 |
| Patient 1 | 23 | 2 | 1.0346 | 990150 | 2475.35 |
| Patient 1 | 7 | 1 | 1.3912 | 123185 | 2590.99 |
| Patient 1 | 7 | 0 | 2.2543 | 447820 | 1363.25 |
| Patient 1 | 14 | 1 | 1.1461 | 482767 | 2408.45 |
| Patient 1 | 9 | 1 | 1.1990 | 420275 | 2976.57 |
| Patient 1 | 11 | 1 | 1.3701 | 774716 | 1889.26 |
| Patient 1 | 20 | 2 | 1.3030 | 1031207 | 2289.08 |
| Patient 1 | 24 | 2 | 0.7295 | 419827 | 2406.18 |
| Patient 1 | 24 | 4 | 1.1106 | 1238619 | 2450.90 |
| Patient 1 | 15 | 1 | 1.6677 | 283097 | 1870.84 |
| Patient 1 | 3 | 0 | 173875323.8880 | 58672 | 1738.99 |
| Patient 1 | 13 | 2 | 1.5348 | 646031 | 2433.35 |
| Patient 1 | 4 | 0 | 1.9779 | 159376 | 2035.12 |
| Patient 1 | 30 | 3 | 1.6861 | 927651 | 2052.57 |
| Patient 1 | 10 | 0 | 1.7051 | 449237 | 2243.52 |
| Patient 1 | 38 | 1 | 1.1519 | 701689 | 2163.62 |
| Patient 1 | 13 | 0 | 2.2914 | 307237 | 2222.85 |
| Patient 1 | 26 | 2 | 1.3053 | 963180 | 2561.22 |
| Patient 1 | 26 | 4 | 1.4786 | 745341 | 2394.70 |
| Patient 1 | 9 | 0 | 2.9394 | 428650 | 1836.88 |
| Patient 1 | 5 | 0 | 1.0103 | 98785 | 2099.12 |
| Patient 1 | 27 | 3 | 1.8216 | 875450 | 2907.96 |
| Patient 1 | 19 | 2 | 1.2500 | 707169 | 2624.37 |
| Patient 1 | 24 | 3 | 1.3563 | 918584 | 2645.53 |
| Patient 1 | 9 | 0 | 1.8391 | 487456 | 2578.81 |
| Patient 1 | 11 | 0 | 1.9155 | 290449 | 2449.35 |
| Patient 1 | 10 | 0 | 1.2108 | 327928 | 1711.33 |
| Patient 1 | 3 | 0 | 179500794.0487 | 388594 | 2278.89 |
| Patient 1 | 10 | 0 | 2.7198 | 580035 | 2146.93 |
| Patient 1 | 15 | 0 | 1.0554 | 797298 | 1753.25 |
| Patient 1 | 14 | 2 | 1.0219 | 494727 | 2173.27 |
| Patient 1 | 8 | 1 | 1.4331 | 493420 | 1641.96 |
| Patient 1 | 8 | 1 | 2.0786 | 249448 | 2822.54 |
| Patient 1 | 13 | 1 | 1.8520 | 279392 | 2408.09 |
| Patient 1 | 40 | 1 | 1.3435 | 1153473 | 3042.74 |
| Patient 1 | 50 | 9 | 1.9709 | 1581000 | 3506.47 |
| Patient 1 | 14 | 0 | 0.9218 | 582802 | 3047.91 |
| Patient 1 | 57 | 8 | 1.3164 | 1684625 | 2565.75 |
| Patient 1 | 27 | 5 | 1.4343 | 923842 | 2676.26 |
| Patient 1 | 3 | 0 | 89602489.6838 | 42350 | 3286.47 |
| Patient 1 | 22 | 3 | 0.6489 | 738613 | 2503.12 |
| Patient 1 | 19 | 2 | 1.7903 | 212491 | 2193.12 |
| Patient 1 | 7 | 0 | 2.1833 | 126542 | 1613.08 |
| Patient 1 | 34 | 5 | 1.3160 | 1237194 | 2274.50 |
| Patient 1 | 19 | 2 | 1.2756 | 395235 | 2007.68 |
| Patient 1 | 9 | 0 | 1.4105 | 293749 | 1701.49 |
| Patient 1 | 41 | 1 | 1.4660 | 1063343 | 2480.24 |
| Patient 1 | 32 | 2 | 1.0853 | 838082 | 2476.28 |
| Patient 1 | 7 | 1 | 1.3889 | 102588 | 2426.73 |
| Patient 1 | 22 | 0 | 1.6024 | 674425 | 2908.27 |
| Patient 1 | 18 | 2 | 1.2573 | 322508 | 2431.13 |
| Patient 1 | 37 | 6 | 1.5680 | 759785 | 2392.32 |
| Patient 1 | 6 | 1 | 0.6991 | 220773 | 2043.00 |
| Patient 1 | 24 | 3 | 1.3546 | 909948 | 2530.19 |
| Patient 1 | 7 | 2 | 0.6728 | 779458 | 1603.40 |
| Patient 1 | 16 | 2 | 1.1514 | 537002 | 2579.58 |
| Patient 1 | 12 | 1 | 1.2083 | 619377 | 2347.10 |
| Patient 1 | 26 | 4 | 1.3996 | 585361 | 2299.74 |
| Patient 1 | 31 | 0 | 1.4204 | 1202778 | 2544.77 |
| Patient 1 | 22 | 4 | 1.3779 | 966963 | 2147.39 |
| Patient 1 | 33 | 4 | 1.2443 | 1311596 | 2379.73 |
| Patient 1 | 19 | 1 | 1.2405 | 485133 | 2233.69 |
| Patient 1 | 15 | 0 | 1.4306 | 626138 | 2499.18 |
| Patient 1 | 11 | 0 | 1.7937 | 502800 | 2336.55 |
| Patient 1 | 39 | 1 | 1.3909 | 715239 | 2240.76 |
| Patient 2 | 5 | 0 | 2.9751 | 176775 | 1823.24 |
| Patient 2 | 23 | 3 | 1.6710 | 932760 | 2695.67 |
| Patient 2 | 15 | 2 | 1.8727 | 313789 | 2079.32 |
| Patient 2 | 16 | 2 | 2.4343 | 278657 | 2837.54 |
| Patient 2 | 14 | 0 | 1.1920 | 796958 | 1935.10 |
| Patient 2 | 8 | 0 | 2.7516 | 183014 | 2261.95 |
| Patient 2 | 7 | 0 | 1.8551 | 88027 | 2029.62 |
| Patient 2 | 16 | 1 | 1.7368 | 574194 | 2448.92 |
| Patient 2 | 8 | 1 | 1.4069 | 55816 | 1832.99 |
| Patient 2 | 14 | 2 | 2.0567 | 722136 | 2946.74 |
| Patient 2 | 5 | 1 | 1.1075 | 76066 | 1930.28 |
| Patient 2 | 11 | 0 | 1.3672 | 178097 | 2274.49 |
| Patient 2 | 4 | 0 | 8.4031 | 99299 | 2798.87 |
| Patient 2 | 7 | 1 | 1.1332 | 217098 | 1932.22 |
| Patient 2 | 13 | 0 | 0.8898 | 407372 | 1856.37 |
| Patient 2 | 28 | 3 | 1.2980 | 663787 | 2483.16 |
| Patient 2 | 14 | 2 | 1.8202 | 645408 | 2591.29 |
| Patient 2 | 12 | 1 | 1.1651 | 359885 | 2040.65 |
| Patient 2 | 13 | 1 | 0.8276 | 625780 | 2074.84 |
| Patient 2 | 14 | 0 | 1.7129 | 514455 | 2714.63 |
| Patient 2 | 22 | 3 | 1.4214 | 705633 | 2014.81 |
| Patient 2 | 10 | 1 | 1.9905 | 514108 | 2149.75 |
| Patient 2 | 9 | 1 | 1.9237 | 396750 | 2702.29 |
| Patient 2 | 3 | 1 | 3.5520 | 65744 | 2508.81 |
| Patient 2 | 13 | 1 | 1.8914 | 737353 | 2142.33 |
| Patient 2 | 8 | 0 | 2.5056 | 237964 | 2390.66 |
| Patient 2 | 9 | 0 | 1.5138 | 236341 | 1490.50 |
| Patient 2 | 12 | 2 | 1.5442 | 305354 | 2305.96 |
| Patient 2 | 2 | 0 | 4.1026 | 23828 | 2439.03 |
| Patient 2 | 12 | 1 | 2.4907 | 220918 | 2134.38 |
| Patient 2 | 23 | 1 | 1.2132 | 403731 | 1987.57 |
| Patient 2 | 9 | 1 | 1.0373 | 403840 | 2235.90 |
| Patient 2 | 2 | 0 | 34416.2089 | 25896 | 2480.58 |
| Patient 2 | 7 | 1 | 1.5045 | 239144 | 1519.25 |
| Patient 2 | 20 | 2 | 1.9993 | 631395 | 2496.11 |
| Patient 2 | 4 | 0 | 13.1418 | 256185 | 1050.69 |
| Patient 2 | 24 | 2 | 2.5794 | 625919 | 2311.49 |
| Patient 2 | 9 | 1 | 2.1267 | 251883 | 2686.76 |
| Patient 2 | 13 | 0 | 1.5319 | 756553 | 2065.40 |
| Patient 2 | 17 | 2 | 2.0323 | 640192 | 2105.12 |
| Patient 2 | 22 | 3 | 1.7635 | 860450 | 2561.16 |
| Patient 2 | 10 | 0 | 2.6159 | 478207 | 2297.21 |
| Patient 2 | 7 | 1 | 2.2271 | 347534 | 1905.55 |
| Patient 2 | 16 | 3 | 1.2867 | 544973 | 2632.96 |
| Patient 2 | 9 | 2 | 1.4159 | 366404 | 1847.04 |
| Patient 2 | 8 | 0 | 1.4128 | 108547 | 2159.89 |
| Patient 2 | 2 | 0 | 1.8203 | 64675 | 924.26 |
| Patient 2 | 9 | 0 | 4.1829 | 138233 | 3658.68 |
| Patient 2 | 17 | 1 | 1.2935 | 537839 | 2523.86 |
| Patient 2 | 28 | 6 | 1.3298 | 687466 | 2988.05 |
| Patient 2 | 16 | 0 | 1.5634 | 446556 | 2102.44 |
| Patient 2 | 14 | 1 | 1.7722 | 325986 | 1924.82 |
| Patient 2 | 23 | 3 | 1.4431 | 1077134 | 2621.44 |
| Patient 2 | 23 | 2 | 1.6126 | 811250 | 2130.76 |
| Patient 2 | 11 | 1 | 2.4036 | 145673 | 2344.73 |
| Patient 2 | 11 | 3 | 6.0189 | 685111 | 2825.84 |
| Patient 2 | 9 | 0 | 0.8177 | 228811 | 2305.56 |
| Patient 2 | 15 | 0 | 1.3236 | 672140 | 2318.01 |
| Patient 2 | 12 | 2 | 1.0378 | 211998 | 2057.85 |
| Patient 2 | 8 | 0 | 1.5287 | 73601 | 2361.08 |
| Patient 2 | 28 | 4 | 0.8562 | 709937 | 2569.88 |
| Patient 2 | 20 | 2 | 0.8162 | 708534 | 2685.31 |
| Patient 2 | 19 | 1 | 1.3267 | 573391 | 2511.43 |
| Patient 2 | 16 | 3 | 1.3078 | 541319 | 2372.90 |
| Patient 2 | 10 | 0 | 1.5262 | 327484 | 2963.01 |
| Patient 2 | 20 | 2 | 1.5444 | 595385 | 2979.54 |
| Patient 3 | 8 | 1 | 1.5633 | 311862 | 1797.69 |
| Patient 3 | 17 | 1 | 1.3169 | 729374 | 2351.92 |
| Patient 3 | 6 | 1 | 1.3271 | 54086 | 2042.35 |
| Patient 3 | 9 | 0 | 1.8944 | 196024 | 1791.35 |
| Patient 3 | 26 | 4 | 1.1648 | 706919 | 2492.53 |
| Patient 3 | 28 | 4 | 2.0513 | 810185 | 1980.44 |
| Patient 3 | 10 | 2 | 1.6768 | 327661 | 2434.62 |
| Patient 3 | 5 | 0 | 3.0582 | 227897 | 2607.74 |
| Patient 3 | 7 | 0 | 0.5368 | 722589 | 1747.40 |
| Patient 3 | 17 | 1 | 1.7597 | 1123490 | 2155.11 |
| Patient 3 | 21 | 2 | 1.7252 | 612810 | 2269.21 |
| Patient 3 | 24 | 5 | 1.4681 | 688732 | 2590.48 |
| Patient 3 | 21 | 4 | 1.7558 | 1003678 | 2656.24 |
| Patient 3 | 15 | 2 | 1.4460 | 726433 | 2413.72 |
| Patient 3 | 14 | 1 | 2.0333 | 352361 | 1715.78 |
| Patient 3 | 12 | 2 | 1.5342 | 392381 | 1845.58 |
| Patient 3 | 17 | 3 | 1.5131 | 409121 | 1790.45 |
| Patient 3 | 14 | 1 | 1.4573 | 658654 | 1870.40 |
| Patient 3 | 18 | 1 | 1.6278 | 1048277 | 1938.19 |
| Patient 3 | 22 | 1 | 2.1677 | 948075 | 2312.23 |
| Patient 3 | 8 | 1 | 1.6963 | 311009 | 1643.96 |
| Patient 3 | 13 | 0 | 1.6522 | 810528 | 2244.11 |
| Patient 3 | 17 | 0 | 1.0748 | 219593 | 1866.14 |
| Patient 3 | 14 | 2 | 2.0502 | 557408 | 2076.83 |
| Patient 3 | 3 | 0 | 2.4880 | 128917 | 2177.48 |
| Patient 3 | 11 | 1 | 1.3818 | 493867 | 2189.54 |
| Patient 3 | 19 | 3 | 2.0095 | 1385293 | 1996.55 |
| Patient 3 | 18 | 3 | 1.2862 | 709281 | 2030.29 |
| Patient 3 | 10 | 1 | 1.4218 | 395772 | 1810.99 |
| Patient 3 | 12 | 1 | 2.2727 | 373351 | 1523.72 |
| Patient 3 | 5 | 0 | 1.1734 | 243045 | 1608.42 |
| Patient 3 | 22 | 4 | 1.5822 | 898082 | 2869.13 |
| Patient 3 | 11 | 0 | 1.5466 | 555529 | 1778.53 |
| Patient 3 | 5 | 0 | 3.3905 | 154312 | 2386.86 |
| Patient 3 | 11 | 0 | 1.7067 | 492882 | 1659.20 |
| Patient 3 | 26 | 1 | 1.9230 | 896845 | 2521.23 |
| Patient 3 | 7 | 0 | 1.1047 | 633739 | 1348.18 |
| Patient 3 | 10 | 1 | 1.4219 | 412384 | 1585.83 |
| Patient 3 | 21 | 1 | 1.5626 | 772549 | 2311.30 |
| Patient 3 | 6 | 0 | 3.8118 | 306708 | 1932.49 |
| Patient 3 | 14 | 0 | 1.3754 | 1196929 | 2143.82 |
| Patient 3 | 18 | 1 | 1.2175 | 457921 | 2105.21 |
| Patient 3 | 22 | 2 | 1.3191 | 733549 | 2287.55 |
| Patient 3 | 15 | 3 | 1.6482 | 671670 | 1910.32 |
| Patient 3 | 9 | 0 | 1.2058 | 529881 | 1633.93 |
| Patient 3 | 14 | 2 | 1.4045 | 592404 | 2007.55 |
| Patient 3 | 16 | 1 | 0.9135 | 543195 | 1982.94 |
| Patient 3 | 15 | 3 | 1.2610 | 622416 | 2013.37 |
| Patient 3 | 14 | 1 | 1.8045 | 503862 | 1858.05 |
| Patient 3 | 7 | 0 | 1.8211 | 65679 | 2085.13 |
| Patient 3 | 16 | 2 | 1.0930 | 654586 | 2412.27 |
| Patient 3 | 13 | 1 | 1.5149 | 1153261 | 2286.58 |
| Patient 3 | 9 | 1 | 1.3614 | 203015 | 2334.45 |
| Patient 3 | 11 | 0 | 2.0836 | 280443 | 1932.97 |
| Patient 3 | 27 | 4 | 2.7676 | 755133 | 3896.19 |
| Patient 3 | 7 | 1 | 2.7428 | 156912 | 2046.85 |
| Patient 3 | 29 | 4 | 2.4057 | 931854 | 2387.01 |
| Patient 3 | 15 | 2 | 1.7160 | 252202 | 2042.37 |
| Patient 3 | 17 | 3 | 2.1438 | 427468 | 2241.32 |
| Patient 3 | 3 | 1 | 8.6813 | 110495 | 1414.78 |
| Patient 3 | 14 | 2 | 1.2343 | 264313 | 1787.44 |
| Patient 3 | 16 | 1 | 1.5138 | 535244 | 2446.68 |
| Patient 3 | 31 | 1 | 1.5694 | 694618 | 2343.73 |
| Patient 3 | 4 | 0 | 2.0840 | 66154 | 2559.59 |
| Patient 3 | 6 | 0 | 26.0346 | 102031 | 1628.14 |
| Patient 3 | 5 | 0 | 1.6161 | 345543 | 1380.47 |
| Patient 3 | 19 | 3 | 1.6872 | 707234 | 2028.23 |
| Patient 3 | 7 | 0 | 1.2583 | 152094 | 1439.44 |
| Patient 3 | 7 | 0 | 1.8338 | 215202 | 1633.91 |
| Patient 3 | 5 | 0 | 3.0582 | 227897 | 2607.74 |
| Patient 4 | 9 | 0 | 0.8742 | 438479 | 1325.98 |
| Patient 4 | 5 | 0 | 1.4001 | 164219 | 2327.69 |
| Patient 4 | 4 | 0 | 3.0268 | 80558 | 1757.91 |
| Patient 4 | 8 | 1 | 0.5430 | 328377 | 1569.98 |
| Patient 4 | 31 | 4 | 1.4039 | 1238979 | 2752.57 |
| Patient 4 | 11 | 0 | 1.2301 | 543163 | 2113.30 |
| Patient 4 | 21 | 2 | 1.1167 | 541910 | 2025.06 |
| Patient 4 | 7 | 1 | 1.7412 | 199007 | 2364.54 |
| Patient 4 | 5 | 1 | 0.5851 | 359445 | 1481.44 |
| Patient 4 | 36 | 5 | 1.0586 | 1226034 | 2424.16 |
| Patient 4 | 5 | 0 | 5.0690 | 87499 | 2632.69 |
| Patient 4 | 12 | 2 | 0.7476 | 289020 | 2308.68 |
| Patient 4 | 10 | 1 | 2.6821 | 483075 | 2429.67 |
| Patient 4 | 21 | 4 | 1.3256 | 937966 | 2005.37 |
| Patient 4 | 23 | 1 | 1.1112 | 575775 | 2049.46 |
| Patient 4 | 27 | 2 | 1.1301 | 823751 | 2064.41 |
| Patient 4 | 9 | 1 | 1.3632 | 436335 | 1951.45 |
| Patient 4 | 24 | 1 | 0.8089 | 824657 | 2341.85 |
| Patient 4 | 16 | 0 | 1.0466 | 407108 | 1862.82 |
| Patient 4 | 12 | 0 | 2.3241 | 314482 | 2096.93 |
| Patient 4 | 16 | 2 | 1.0104 | 618138 | 1819.81 |
| Patient 4 | 9 | 0 | 1.2815 | 689976 | 1863.67 |
| Patient 4 | 21 | 3 | 1.4867 | 1098816 | 2394.41 |
| Patient 4 | 4 | 0 | 2.2398 | 305632 | 1286.81 |
| Patient 4 | 13 | 2 | 1.1317 | 830048 | 2038.11 |
| Patient 4 | 12 | 0 | 0.7903 | 623339 | 2596.25 |
| Patient 4 | 19 | 0 | 1.1961 | 841675 | 2342.34 |
| Patient 4 | 20 | 2 | 1.2623 | 829945 | 1799.57 |
| Patient 4 | 9 | 0 | 1.8397 | 463336 | 2425.85 |
| Patient 4 | 14 | 1 | 1.4737 | 291591 | 2219.09 |
| Patient 4 | 16 | 3 | 2.1324 | 435107 | 2129.84 |
| Patient 4 | 11 | 0 | 1.4528 | 374782 | 1951.04 |
| Patient 4 | 26 | 1 | 1.3111 | 685500 | 2393.45 |
| Patient 4 | 2 | 1 | 120.1633 | 151430 | 2193.55 |
| Patient 4 | 4 | 0 | 146.9631 | 135726 | 3158.56 |
| Patient 4 | 19 | 3 | 2.6157 | 607855 | 2233.70 |
| Patient 4 | 16 | 2 | 1.3178 | 713262 | 2407.99 |
| Patient 4 | 9 | 1 | 0.9494 | 686802 | 1716.96 |
| Patient 4 | 8 | 2 | 1.1308 | 409083 | 2276.79 |
| Patient 4 | 16 | 2 | 1.2545 | 918535 | 1933.69 |
| Patient 4 | 6 | 1 | 1.0859 | 400651 | 2314.93 |
| Patient 4 | 16 | 1 | 1.8741 | 852664 | 2225.04 |
| Patient 4 | 10 | 1 | 1.4232 | 225808 | 2356.31 |
| Patient 4 | 38 | 6 | 1.0821 | 553102 | 2711.10 |
| Patient 4 | 10 | 2 | 1.7395 | 313816 | 1756.43 |
| Patient 4 | 12 | 2 | 1.7521 | 523238 | 1759.37 |
| Patient 4 | 17 | 2 | 1.6709 | 468183 | 1949.66 |
| Patient 4 | 11 | 0 | 1.6548 | 317337 | 1824.47 |
| Patient 4 | 14 | 0 | 1.1367 | 692323 | 1854.05 |
| Patient 4 | 17 | 3 | 1.6966 | 873855 | 2301.44 |
| Patient 4 | 14 | 0 | 0.9317 | 875232 | 2295.78 |
| Patient 4 | 7 | 1 | 2.1511 | 421426 | 2185.29 |
| Patient 4 | 5 | 2 | 2.9365 | 168366 | 1878.04 |
| Patient 4 | 44 | 4 | 1.5782 | 1644164 | 2720.91 |
| Patient 4 | 15 | 2 | 0.9513 | 987866 | 2114.34 |
| Patient 4 | 12 | 2 | 1.4638 | 802091 | 2194.97 |
| Patient 4 | 31 | 4 | 1.6752 | 747914 | 2850.89 |
| Patient 4 | 17 | 1 | 1.1072 | 594765 | 2436.67 |
| Patient 4 | 7 | 0 | 1.5791 | 324246 | 2233.23 |
| Patient 4 | 21 | 1 | 1.3341 | 798221 | 2310.88 |
| Patient 4 | 12 | 2 | 1.3785 | 388764 | 2012.34 |
| Patient 4 | 9 | 2 | 1.5721 | 301611 | 2540.84 |
| Patient 4 | 20 | 3 | 1.4527 | 889408 | 2339.46 |
| Patient 4 | 26 | 5 | 1.6266 | 976355 | 2511.46 |
| Patient 4 | 9 | 0 | 1.0643 | 534650 | 1348.61 |
| Patient 4 | 11 | 1 | 1.2650 | 242075 | 2219.47 |
| Patient 4 | 17 | 1 | 1.5091 | 608099 | 2990.69 |
| Patient 4 | 9 | 0 | 3.2641 | 407041 | 1667.51 |
| Patient 4 | 20 | 0 | 1.6472 | 860880 | 2551.02 |
| Patient 5 | 12 | 1 | 1.5388 | 397757 | 1681.62 |
| Patient 5 | 22 | 2 | 1.1609 | 1267954 | 2526.20 |
| Patient 5 | 30 | 2 | 1.0601 | 902734 | 2221.43 |
| Patient 5 | 31 | 3 | 1.1080 | 1088730 | 2435.28 |
| Patient 5 | 17 | 1 | 0.8975 | 645116 | 1827.84 |
| Patient 5 | 16 | 1 | 1.0800 | 834672 | 2121.89 |
| Patient 5 | 10 | 2 | 1.7582 | 426234 | 1769.79 |
| Patient 5 | 31 | 3 | 1.9778 | 1075847 | 2543.42 |
| Patient 5 | 18 | 0 | 1.2201 | 918940 | 2076.57 |
| Patient 5 | 11 | 1 | 1.1481 | 643308 | 2188.50 |
| Patient 5 | 12 | 0 | 2.2444 | 305059 | 2429.56 |
| Patient 5 | 38 | 4 | 1.5629 | 1239576 | 2284.19 |
| Patient 5 | 18 | 1 | 1.1285 | 650527 | 2056.61 |
| Patient 5 | 8 | 1 | 1.5601 | 436335 | 2012.20 |
| Patient 5 | 22 | 2 | 1.6695 | 1062720 | 2192.31 |
| Patient 5 | 14 | 2 | 1.3355 | 445618 | 1749.14 |
| Patient 5 | 4 | 0 | 1.5258 | 149479 | 1131.63 |
| Patient 5 | 29 | 3 | 1.0374 | 720648 | 2159.40 |
| Patient 5 | 15 | 2 | 1.6522 | 402708 | 1827.95 |
| Patient 5 | 17 | 3 | 1.5338 | 565853 | 2231.09 |
| Patient 5 | 19 | 1 | 1.1616 | 497342 | 1773.83 |
| Patient 5 | 35 | 3 | 1.6223 | 1449345 | 2812.95 |
| Patient 5 | 22 | 4 | 2.5174 | 526311 | 2316.22 |
| Patient 5 | 39 | 5 | 1.4479 | 1032643 | 2600.25 |
| Patient 5 | 11 | 1 | 2.9467 | 476862 | 1931.04 |
| Patient 5 | 40 | 5 | 1.2747 | 1386497 | 2471.84 |
| Patient 5 | 22 | 4 | 0.2416 | 693718 | 1859.68 |
| Patient 5 | 31 | 3 | 1.0820 | 901567 | 2487.13 |
| Patient 5 | 14 | 1 | 0.7534 | 548488 | 1747.85 |
| Patient 5 | 25 | 5 | 1.4605 | 1239476 | 2506.93 |
| Patient 5 | 23 | 2 | 1.6852 | 900056 | 2264.28 |
| Patient 5 | 19 | 1 | 1.9200 | 792307 | 1931.53 |
| Patient 5 | 29 | 6 | 1.2725 | 779381 | 2281.52 |
| Patient 5 | 33 | 5 | 1.3644 | 909363 | 2437.15 |
| Patient 5 | 17 | 1 | 0.2606 | 797143 | 1981.10 |
| Patient 5 | 32 | 3 | 1.2247 | 1190485 | 2364.72 |
| Patient 5 | 16 | 3 | 1.9232 | 568094 | 2613.40 |
| Patient 5 | 6 | 0 | 1.5048 | 161260 | 1245.68 |
| Patient 5 | 32 | 5 | 1.7725 | 1354015 | 2203.16 |
| Patient 5 | 27 | 5 | 1.3608 | 1171650 | 2237.02 |
| Patient 5 | 19 | 4 | 1.7922 | 632310 | 2345.29 |
| Patient 5 | 22 | 3 | 1.1787 | 953158 | 2147.12 |
| Patient 5 | 9 | 2 | 1.6496 | 495671 | 1820.82 |
| Patient 5 | 28 | 0 | 1.6105 | 1133522 | 2748.93 |
| Patient 5 | 3 | 0 | 245607783.6201 | 143756 | 755.50 |
| Patient 5 | 51 | 5 | 2.0107 | 1546285 | 2896.47 |
| Patient 5 | 31 | 2 | 1.3910 | 786821 | 2580.43 |
| Patient 5 | 54 | 9 | 2.0017 | 1448839 | 4321.78 |
| Patient 5 | 72 | 5 | 1.3268 | 2592446 | 3078.92 |
| Patient 5 | 45 | 9 | 1.5151 | 1555992 | 2854.37 |
| Patient 5 | 29 | 5 | 1.3830 | 1031093 | 2243.70 |
| Patient 5 | 23 | 0 | 1.4583 | 697742 | 2652.28 |
| Patient 5 | 18 | 2 | 1.0380 | 617338 | 1950.63 |
| Patient 5 | 13 | 2 | 1.5107 | 527467 | 2331.33 |
| Patient 5 | 44 | 6 | 0.9674 | 1273500 | 2550.45 |
| Patient 5 | 34 | 5 | 1.3901 | 988109 | 2428.39 |
| Patient 5 | 27 | 4 | 1.8396 | 847219 | 2529.99 |
| Patient 5 | 36 | 3 | 1.1729 | 1375681 | 2755.22 |
| Patient 5 | 52 | 5 | 1.3790 | 2275047 | 2816.25 |
| Patient 5 | 45 | 5 | 2.5604 | 1356522 | 3111.65 |
| Patient 5 | 26 | 0 | 1.5135 | 885550 | 2370.78 |
| Patient 5 | 31 | 4 | 1.6463 | 771668 | 2459.99 |
| Patient 5 | 24 | 1 | 1.0402 | 1205670 | 2388.87 |
| Patient 5 | 17 | 3 | 1.3520 | 508335 | 2205.48 |
| Patient 5 | 19 | 0 | 2.1089 | 491377 | 2256.26 |
| Patient 5 | 24 | 1 | 2.0141 | 727127 | 2121.41 |
| Patient 5 | 18 | 3 | 1.2520 | 780766 | 2130.43 |
| Patient 5 | 27 | 1 | 1.3393 | 564350 | 2269.19 |
| Patient 5 | 17 | 3 | 0.9991 | 517418 | 2098.08 |
| Patient 5 | 10 | 1 | 0.6310 | 426359 | 1864.82 |
| Patient 5 | 12 | 1 | 1.5388 | 397757 | 1677.36 |
| Patient 5 | 14 | 1 | 2.0588 | 401579 | 2600.95 |
| Patient 6 | 12 | 0 | 0.9513 | 453265 | 1509.61 |
| Patient 6 | 16 | 2 | 1.1378 | 579522 | 2187.59 |
| Patient 6 | 16 | 2 | 1.2095 | 657269 | 2640.12 |
| Patient 6 | 22 | 0 | 1.7390 | 526368 | 2157.02 |
| Patient 6 | 7 | 0 | 0.6928 | 215261 | 1264.56 |
| Patient 6 | 37 | 5 | 1.7768 | 1350769 | 3278.04 |
| Patient 6 | 12 | 2 | 0.7658 | 560615 | 2028.60 |
| Patient 6 | 7 | 0 | 1.8627 | 85095 | 1369.22 |
| Patient 6 | 5 | 0 | 1.0238 | 188015 | 1537.70 |
| Patient 6 | 6 | 2 | 1.7845 | 247402 | 1495.83 |
| Patient 6 | 22 | 1 | 1.4621 | 723973 | 1956.87 |
| Patient 6 | 5 | 1 | 1.4204 | 237577 | 2178.09 |
| Patient 6 | 7 | 0 | 7.5700 | 281398 | 2168.44 |
| Patient 6 | 24 | 3 | 1.3043 | 1466672 | 2989.46 |
| Patient 6 | 13 | 1 | 1.2521 | 516169 | 2277.71 |
| Patient 6 | 29 | 3 | 1.1920 | 1006217 | 2508.61 |
| Patient 6 | 22 | 1 | 1.4218 | 728220 | 1940.38 |
| Patient 6 | 2 | 1 | 3.2868 | 49777 | 1523.32 |
| Patient 6 | 4 | 0 | 1.9796 | 46225 | 1584.25 |
| Patient 6 | 5 | 0 | 1.2786 | 216507 | 2194.91 |
| Patient 6 | 12 | 1 | 2.1859 | 326207 | 1520.52 |
| Patient 6 | 20 | 1 | 0.8814 | 973870 | 2106.80 |
| Patient 6 | 25 | 3 | 1.7799 | 697232 | 2202.64 |
| Patient 6 | 18 | 3 | 2.8648 | 466577 | 1925.88 |
| Patient 6 | 9 | 0 | 1.2636 | 381872 | 1479.65 |
| Patient 6 | 10 | 0 | 1.3790 | 558874 | 2054.16 |
| Patient 6 | 6 | 0 | 1.7074 | 161528 | 1450.18 |
| Patient 6 | 8 | 0 | 0.6660 | 330517 | 1227.17 |
| Patient 6 | 20 | 3 | 0.8385 | 674360 | 1890.94 |
| Patient 6 | 20 | 1 | 1.8283 | 492816 | 2024.74 |
| Patient 6 | 19 | 1 | 1.7280 | 574207 | 2434.20 |
| Patient 6 | 28 | 3 | 2.1310 | 608646 | 2501.36 |
| Patient 6 | 22 | 2 | 1.8751 | 933857 | 2271.88 |
| Patient 6 | 22 | 1 | 1.4440 | 901482 | 2196.86 |
| Patient 6 | 20 | 1 | 0.8344 | 928924 | 2235.00 |
| Patient 6 | 28 | 4 | 1.2036 | 656931 | 2096.59 |
| Patient 6 | 18 | 0 | 1.9155 | 456464 | 1999.90 |
| Patient 6 | 15 | 1 | 1.8675 | 698227 | 1857.00 |
| Patient 6 | 17 | 1 | 2.7779 | 298982 | 2502.84 |
| Patient 6 | 23 | 3 | 1.3745 | 877206 | 2231.80 |
| Patient 6 | 11 | 2 | 0.9994 | 646210 | 2181.76 |
| Patient 6 | 3 | 1 | 3.3727 | 103071 | 1166.84 |
| Patient 6 | 27 | 1 | 1.2516 | 725327 | 2175.54 |
| Patient 6 | 16 | 1 | 1.4337 | 471885 | 1701.60 |
| Patient 6 | 2 | 0 | 1.3806 | 42998 | 2261.08 |
| Patient 6 | 21 | 3 | 1.3297 | 912800 | 2221.23 |
| Patient 6 | 5 | 0 | 2.8450 | 486435 | 1817.80 |
| Patient 6 | 72 | 9 | 1.7799 | 1590470 | 3111.92 |
| Patient 6 | 20 | 3 | 1.3047 | 520232 | 1989.42 |
| Patient 6 | 8 | 0 | 1.8721 | 111775 | 2723.21 |
| Patient 6 | 19 | 4 | 1.7127 | 965588 | 2159.00 |
| Patient 6 | 24 | 1 | 1.3999 | 533497 | 2027.03 |
| Patient 6 | 16 | 2 | 2.1325 | 446065 | 1899.22 |
| Patient 6 | 17 | 2 | 1.2736 | 674184 | 1941.72 |
| Patient 6 | 29 | 4 | 2.2320 | 613171 | 2429.22 |
| Patient 6 | 17 | 0 | 1.8967 | 267945 | 1923.50 |
| Patient 6 | 8 | 0 | 1.4200 | 295751 | 2289.48 |
| Patient 6 | 30 | 3 | 1.7695 | 940098 | 2443.03 |
| Patient 6 | 25 | 4 | 1.0244 | 633662 | 2236.64 |
| Patient 6 | 43 | 2 | 1.6664 | 1981974 | 3099.22 |
| Patient 6 | 6 | 1 | 1.8756 | 283063 | 2171.06 |
| Patient 6 | 6 | 0 | 1.6115 | 359864 | 1513.47 |
| Patient 6 | 15 | 1 | 1.5632 | 760362 | 2102.29 |
| Patient 6 | 29 | 5 | 1.4629 | 565234 | 2364.84 |
| Patient 6 | 43 | 3 | 1.3571 | 1698748 | 2341.26 |
| Patient 6 | 15 | 1 | 1.7865 | 1029315 | 2159.30 |
| Patient 6 | 26 | 4 | 1.3123 | 655151 | 2265.58 |
| Patient 6 | 12 | 0 | 1.2752 | 522573 | 2096.32 |
| Patient 6 | 14 | 1 | 1.4415 | 546149 | 2396.42 |
| Patient 6 | 11 | 0 | −0.4831 | 662441 | 1568.21 |
| Patient 7 | 6 | 0 | 1.2207 | 107228 | 1790.32 |
| Patient 7 | 20 | 4 | 1.7688 | 825309 | 2301.03 |
| Patient 7 | 15 | 1 | 1.9077 | 475684 | 2432.40 |
| Patient 7 | 8 | 0 | 1.5327 | 295814 | 2059.48 |
| Patient 7 | 19 | 2 | 2.4984 | 644745 | 2614.40 |
| Patient 7 | 25 | 3 | 0.4258 | 693866 | 2512.67 |
| Patient 7 | 4 | 0 | 1.1432 | 201753 | 1879.96 |
| Patient 7 | 25 | 2 | 1.8236 | 1001183 | 2617.14 |
| Patient 7 | 17 | 0 | 1.5384 | 284964 | 2046.87 |
| Patient 7 | 10 | 2 | 0.6131 | 346881 | 1526.50 |
| Patient 7 | 3 | 0 | 2.2560 | 103482 | 928.46 |
| Patient 7 | 58 | 10 | 3.7244 | 1528984 | 3675.48 |
| Patient 7 | 16 | 0 | 1.1845 | 648402 | 2196.05 |
| Patient 7 | 10 | 1 | 1.1910 | 314328 | 1744.63 |
| Patient 7 | 21 | 4 | 1.0040 | 564365 | 2029.51 |
| Patient 7 | 15 | 0 | 1.6809 | 732764 | 2412.50 |
| Patient 7 | 11 | 1 | 2.8280 | 192119 | 3020.60 |
| Patient 7 | 6 | 1 | 1.1975 | 225866 | 1844.49 |
| Patient 7 | 14 | 2 | 1.8158 | 768163 | 2387.64 |
| Patient 7 | 7 | 0 | 3.8374 | 399961 | 2463.62 |
| Patient 7 | 8 | 0 | 1.1950 | 320928 | 1730.61 |
| Patient 7 | 23 | 1 | 1.1298 | 859519 | 2042.16 |
| Patient 7 | 31 | 3 | 2.1736 | 809900 | 3308.22 |
| Patient 7 | 23 | 1 | 3.7761 | 1260875 | 2253.01 |
| Patient 7 | 5 | 0 | 2.4076 | 228668 | 1728.10 |
| Patient 7 | 19 | 2 | 1.7439 | 634005 | 2208.23 |
| Patient 7 | 13 | 2 | 1.6188 | 367590 | 1748.78 |
| Patient 7 | 8 | 0 | 1.0010 | 134577 | 2034.28 |
| Patient 7 | 17 | 1 | 3.6356 | 544689 | 2311.96 |
| Patient 7 | 33 | 2 | 2.3869 | 1037047 | 2694.57 |
| Patient 7 | 27 | 2 | 1.6359 | 708253 | 2338.35 |
| Patient 7 | 19 | 0 | 2.8174 | 651746 | 2458.42 |
| Patient 7 | 22 | 1 | 2.3405 | 1385723 | 2426.37 |
| Patient 7 | 9 | 0 | 1.6711 | 535296 | 1885.40 |
| Patient 7 | 18 | 1 | 2.2074 | 808818 | 2161.83 |
| Patient 7 | 16 | 0 | 2.0754 | 1067886 | 2085.45 |
| Patient 7 | 11 | 0 | 7.5911 | 390014 | 2622.39 |
| Patient 7 | 14 | 0 | 1.5354 | 502401 | 2122.40 |
| Patient 7 | 4 | 0 | 2.0029 | 118922 | 2262.11 |
| Patient 7 | 17 | 0 | 1.2272 | 725569 | 1622.24 |
| Patient 7 | 14 | 0 | 1.5735 | 465463 | 1935.83 |
| Patient 7 | 27 | 3 | 0.7575 | 646503 | 1993.10 |
| Patient 7 | 20 | 3 | 2.0709 | 764366 | 2179.66 |
| Patient 7 | 7 | 0 | 2.0612 | 136038 | 1890.87 |
| Patient 7 | 8 | 1 | 2.2745 | 357193 | 1546.25 |
| Patient 7 | 10 | 0 | 1.9338 | 459291 | 1774.19 |
| Patient 7 | 10 | 0 | 1.5437 | 190160 | 2250.38 |
| Patient 7 | 14 | 1 | 1.2443 | 296747 | 1521.99 |
| Patient 7 | 3 | 1 | 3.3449 | 31179 | 1928.99 |
| Patient 7 | 18 | 1 | 2.6153 | 1315996 | 2366.49 |
| Patient 7 | 12 | 1 | 1.5097 | 827284 | 1874.18 |
| Patient 7 | 8 | 0 | 1.4105 | 643054 | 1671.85 |
| Patient 7 | 18 | 1 | 3.0445 | 485166 | 2379.06 |
| Patient 7 | 18 | 1 | 1.9060 | 511403 | 1984.58 |
| Patient 7 | 15 | 2 | 1.4443 | 643310 | 1938.93 |
| Patient 7 | 8 | 0 | 1.3918 | 133135 | 1326.50 |
| Patient 7 | 12 | 1 | 1.8042 | 294372 | 1745.35 |
| Patient 7 | 35 | 1 | 1.3829 | 1016579 | 2118.01 |
| Patient 7 | 24 | 2 | 1.8677 | 894379 | 1947.36 |
| Patient 7 | 29 | 3 | 0.9897 | 1131829 | 2394.43 |
| Patient 7 | 4 | 0 | 5.6552 | 127814 | 2136.57 |
| Patient 7 | 15 | 3 | 2.8595 | 550972 | 2215.45 |
| Patient 7 | 20 | 3 | 0.6505 | 619545 | 2062.46 |
| Patient 7 | 10 | 1 | 1.1652 | 196890 | 1546.17 |
| Patient 7 | 5 | 0 | 2.9465 | 86823 | 1510.71 |
| Patient 7 | 8 | 1 | 1.3692 | 163396 | 2107.37 |
| Patient 7 | 22 | 1 | 2.3405 | 1385723 | 2426.37 |
| Patient 8 | 31 | 4 | 1.8951 | 1251660 | 2403.13 |
| Patient 8 | 24 | 5 | 1.7325 | 846406 | 2707.08 |
| Patient 8 | 14 | 1 | 1.2858 | 469156 | 2047.16 |
| Patient 8 | 11 | 1 | 1.7423 | 512282 | 2119.57 |
| Patient 8 | 20 | 0 | 2.7247 | 569124 | 3118.02 |
| Patient 8 | 20 | 2 | 1.7353 | 1158094 | 2220.07 |
| Patient 8 | 3 | 1 | 36129032.1435 | 53216 | 2645.21 |
| Patient 8 | 22 | 3 | 1.4646 | 835994 | 2305.43 |
| Patient 8 | 32 | 7 | 2.1949 | 1268743 | 3038.88 |
| Patient 8 | 20 | 0 | 1.8890 | 543914 | 2179.97 |
| Patient 8 | 18 | 3 | 1.8853 | 703858 | 1739.66 |
| Patient 8 | 8 | 0 | 1.3379 | 364441 | 1379.62 |
| Patient 8 | 18 | 3 | 1.5394 | 763980 | 2203.40 |
| Patient 8 | 13 | 0 | 1.4144 | 1182572 | 1862.11 |
| Patient 8 | 38 | 5 | 2.1391 | 1337582 | 2384.71 |
| Patient 8 | 11 | 1 | 1.3133 | 537823 | 1760.38 |
| Patient 8 | 38 | 5 | 1.5964 | 1019647 | 2428.39 |
| Patient 8 | 15 | 2 | 1.0992 | 513005 | 1990.07 |
| Patient 8 | 12 | 0 | 6.3490 | 374680 | 1693.61 |
| Patient 8 | 9 | 1 | 3.7167 | 377181 | 1903.07 |
| Patient 8 | 19 | 2 | 6.7515 | 160126 | 2727.81 |
| Patient 8 | 10 | 0 | 1.4365 | 139737 | 1913.01 |
| Patient 8 | 15 | 2 | 1.5071 | 494596 | 2019.63 |
| Patient 8 | 2 | 0 | 190891732.5906 | 178162 | 1411.81 |
| Patient 8 | 6 | 0 | 3.5465 | 171728 | 2747.63 |
| Patient 8 | 23 | 2 | 5.3594 | 379762 | 3465.27 |
| Patient 8 | 11 | 1 | 2.7777 | 282474 | 2903.28 |
| Patient 8 | 16 | 0 | 1.4999 | 485076 | 1738.68 |
| Patient 8 | 4 | 0 | 17.7675 | 84525 | 1898.80 |
| Patient 8 | 9 | 0 | 2.8917 | 211034 | 2883.27 |
| Patient 8 | 25 | 0 | 2.1750 | 520873 | 2498.95 |
| Patient 8 | 13 | 1 | 2.4227 | 655055 | 2101.28 |
| Patient 8 | 23 | 1 | 1.5363 | 1026005 | 2236.46 |
| Patient 8 | 18 | 1 | 3.0188 | 345638 | 2606.99 |
| Patient 8 | 12 | 1 | 1.0787 | 308510 | 1824.33 |
| Patient 8 | 16 | 0 | 0.9118 | 508924 | 1968.89 |
| Patient 8 | 9 | 0 | 1.3442 | 185085 | 1925.30 |
| Patient 8 | 45 | 6 | 1.6010 | 1042541 | 2498.88 |
| Patient 8 | 16 | 2 | 1.1618 | 451900 | 1999.11 |
| Patient 8 | 29 | 6 | 1.3437 | 1094306 | 2482.59 |
| Patient 8 | 10 | 0 | 2.0510 | 396621 | 2178.59 |
| Patient 8 | 20 | 1 | 1.5119 | 1182879 | 1937.14 |
| Patient 8 | 23 | 4 | 1.2524 | 730625 | 2798.00 |
| Patient 8 | 22 | 1 | 1.9750 | 714571 | 2536.94 |
| Patient 8 | 14 | 0 | 1.0340 | 560883 | 2118.81 |
| Patient 8 | 29 | 2 | 1.5991 | 1094034 | 2555.43 |
| Patient 8 | 26 | 3 | 2.5473 | 812058 | 2540.90 |
| Patient 8 | 42 | 5 | 1.1962 | 1341606 | 2486.67 |
| Patient 8 | 40 | 0 | 1.1319 | 1517307 | 2577.04 |
| Patient 8 | 19 | 0 | 1.2669 | 480952 | 2352.74 |
| Patient 8 | 2 | 0 | 123011521.6183 | 12782 | 2802.47 |
| Patient 8 | 7 | 1 | 2.4987 | 314122 | 2029.66 |
| Patient 8 | 5 | 0 | 1.5130 | 69383 | 1835.59 |
| Patient 8 | 33 | 1 | 1.1646 | 648858 | 2232.21 |
| Patient 8 | 5 | 0 | 8.1737 | 111168 | 1948.66 |
| Patient 8 | 16 | 2 | 1.6322 | 450585 | 2506.31 |
| Patient 8 | 21 | 0 | 1.5005 | 639305 | 2267.09 |
| Patient 8 | 8 | 0 | 2.0235 | 405069 | 2064.02 |
| Patient 8 | 15 | 0 | 2.0420 | 439836 | 2338.09 |
| Patient 8 | 5 | 0 | 2.2196 | 101014 | 3323.90 |
| Patient 8 | 28 | 5 | 1.3983 | 689536 | 2487.55 |
| Patient 8 | 5 | 0 | 2.2671 | 70434 | 1581.20 |
| Patient 8 | 12 | 1 | 1.5941 | 1297311 | 2216.68 |
| Patient 8 | 9 | 1 | 3.7167 | 377181 | 1903.85 |
| Patient 8 | 5 | 0 | 6.1594 | 93775 | 2010.43 |
| Patient 9 | 4 | 0 | 2.0649 | 423107 | 1421.35 |
| Patient 9 | 1 | 0 | 452421384.3995 | 142201 | 2217.00 |
| Patient 9 | 12 | 1 | 1.9570 | 377936 | 2557.91 |
| Patient 9 | 6 | 1 | 1.2234 | 148915 | 1773.48 |
| Patient 9 | 2 | 1 | 67731497.0319 | 323857 | 1227.83 |
| Patient 9 | 12 | 2 | 1.2483 | 377662 | 1747.93 |
| Patient 9 | 8 | 0 | 1.4559 | 396199 | 1502.61 |
| Patient 9 | 15 | 3 | 1.9207 | 327842 | 2208.85 |
| Patient 9 | 11 | 1 | 1.3774 | 340300 | 1828.25 |
| Patient 9 | 16 | 2 | 1.3165 | 482229 | 2056.08 |
| Patient 9 | 7 | 0 | 1.1154 | 572368 | 1522.27 |
| Patient 9 | 7 | 0 | 1.8380 | 380693 | 1819.08 |
| Patient 9 | 6 | 0 | 1.4128 | 439758 | 2089.27 |
| Patient 9 | 16 | 0 | 1.9991 | 251332 | 2114.19 |
| Patient 9 | 10 | 1 | 1.0094 | 316703 | 2603.49 |
| Patient 9 | 20 | 2 | 1.5422 | 525492 | 2904.40 |
| Patient 9 | 13 | 0 | 1.6903 | 813095 | 2417.08 |
| Patient 9 | 6 | 1 | 3.2703 | 317195 | 1823.77 |
| Patient 9 | 16 | 2 | 1.3665 | 495241 | 2727.75 |
| Patient 9 | 11 | 0 | 1.5395 | 578741 | 2146.73 |
| Patient 9 | 3 | 0 | 6.0684 | 95570 | 2258.04 |
| Patient 9 | 19 | 3 | 1.3850 | 611104 | 2435.06 |
| Patient 9 | 6 | 1 | 5.2816 | 207790 | 1763.74 |
| Patient 9 | 16 | 2 | 1.5212 | 795057 | 2230.06 |
| Patient 9 | 21 | 3 | 1.9013 | 365427 | 2374.36 |
| Patient 9 | 5 | 0 | 7.7200 | 431477 | 1571.14 |
| Patient 9 | 13 | 1 | 1.8293 | 410552 | 2200.30 |
| Patient 9 | 4 | 0 | 4.7667 | 191906 | 1276.04 |
| Patient 9 | 11 | 1 | 1.2052 | 439793 | 1956.11 |
| Patient 9 | 13 | 3 | 1.2229 | 568548 | 2270.23 |
| Patient 9 | 36 | 4 | 1.0513 | 1750805 | 2399.20 |
| Patient 9 | 17 | 2 | 1.4389 | 671998 | 2302.72 |
| Patient 9 | 24 | 1 | 3.0101 | 594218 | 3142.48 |
| Patient 9 | 12 | 1 | 2.7532 | 168021 | 2229.00 |
| Patient 9 | 12 | 0 | 2.8522 | 243448 | 4472.82 |
| Patient 9 | 19 | 3 | 0.6383 | 978611 | 2046.49 |
| Patient 9 | 47 | 6 | 1.6349 | 1320936 | 2683.17 |
| Patient 9 | 9 | 0 | 2.3025 | 768046 | 2448.41 |
| Patient 9 | 20 | 3 | 2.3056 | 996294 | 2321.33 |
| Patient 9 | 10 | 0 | 0.6375 | 1092440 | 1982.96 |
| Patient 9 | 27 | 2 | 1.9721 | 707773 | 2423.72 |
| Patient 9 | 10 | 1 | 1.0198 | 639995 | 1799.98 |
| Patient 9 | 13 | 2 | 1.5233 | 524964 | 1853.37 |
| Patient 9 | 18 | 2 | 1.3224 | 1033025 | 2227.70 |
| Patient 9 | 14 | 1 | 1.3730 | 1017184 | 1879.14 |
| Patient 9 | 17 | 2 | 2.0520 | 751394 | 2457.98 |
| Patient 9 | 15 | 0 | 1.0389 | 523111 | 2366.71 |
| Patient 9 | 16 | 1 | 1.0765 | 572374 | 2850.94 |
| Patient 9 | 20 | 1 | 1.9907 | 702034 | 2413.61 |
| Patient 9 | 18 | 1 | 1.2566 | 1167483 | 2258.75 |
| Patient 9 | 13 | 3 | 1.6243 | 465660 | 1923.25 |
| Patient 9 | 20 | 1 | 1.2922 | 1005159 | 2261.83 |
| Patient 9 | 19 | 1 | 1.8205 | 703451 | 2274.39 |
| Patient 9 | 16 | 1 | 0.5726 | 953925 | 2311.01 |
| Patient 9 | 23 | 4 | 1.5292 | 1030409 | 2206.84 |
| Patient 9 | 13 | 2 | 1.2612 | 552936 | 2049.63 |
| Patient 9 | 12 | 2 | 1.7849 | 615951 | 1779.25 |
| Patient 9 | 9 | 1 | 1.1219 | 798213 | 1749.42 |
| Patient 9 | 21 | 3 | 0.7969 | 569318 | 1976.24 |
| Patient 9 | 5 | 0 | 2.0450 | 250801 | 1395.90 |
| Patient 9 | 15 | 2 | 1.4976 | 679944 | 2182.73 |
| Patient 9 | 10 | 0 | 1.2418 | 900483 | 1597.38 |
| Patient 9 | 15 | 1 | 1.2957 | 783233 | 2001.42 |
| Patient 9 | 22 | 2 | 1.2992 | 1034942 | 2098.35 |
| Patient 9 | 20 | 3 | 1.6713 | 885377 | 2430.73 |
| Patient 9 | 18 | 1 | 1.4738 | 612204 | 1974.92 |
| Patient 9 | 31 | 3 | 1.7550 | 1326800 | 2503.17 |
| Patient 9 | 26 | 5 | 1.2858 | 827708 | 2148.17 |
| Patient 9 | 20 | 1 | 1.4826 | 622081 | 2091.44 |
| Patient 9 | 11 | 1 | 1.2900 | 340300 | 1852.03 |
| Patient 9 | 23 | 0 | 1.1436 | 889668 | 2616.29 |
| Patient 10 | 20 | 3 | 1.3315 | 647537 | 2112.84 |
| Patient 10 | 19 | 0 | 1.3999 | 942403 | 2030.46 |
| Patient 10 | 27 | 6 | 2.1732 | 608135 | 2414.82 |
| Patient 10 | 12 | 2 | 2.4560 | 678970 | 2494.63 |
| Patient 10 | 10 | 0 | 1.6458 | 457273 | 1785.65 |
| Patient 10 | 9 | 0 | 1.8418 | 104382 | 1495.54 |
| Patient 10 | 13 | 2 | 1.4565 | 761343 | 1940.43 |
| Patient 10 | 32 | 5 | 2.0851 | 1114015 | 2262.41 |
| Patient 10 | 10 | 1 | 0.9470 | 303450 | 2231.61 |
| Patient 10 | 12 | 2 | 2.1492 | 565555 | 2074.43 |
| Patient 10 | 12 | 0 | 1.7512 | 320460 | 1966.73 |
| Patient 10 | 14 | 1 | 3.1217 | 427514 | 2211.35 |
| Patient 10 | 16 | 0 | 2.0562 | 614776 | 2049.88 |
| Patient 10 | 14 | 1 | 2.7552 | 491726 | 1978.36 |
| Patient 10 | 26 | 2 | 1.6231 | 848380 | 2568.91 |
| Patient 10 | 8 | 0 | 2.2994 | 205310 | 2284.85 |
| Patient 10 | 7 | 0 | 2.0003 | 60089 | 1693.47 |
| Patient 10 | 17 | 2 | 1.7870 | 909201 | 2104.82 |
| Patient 10 | 34 | 5 | 1.0820 | 1367120 | 2377.97 |
| Patient 10 | 20 | 2 | 0.5973 | 1134755 | 2240.81 |
| Patient 10 | 33 | 4 | 1.3864 | 670010 | 2277.91 |
| Patient 10 | 28 | 4 | 1.2674 | 1267084 | 1677.68 |
| Patient 10 | 17 | 0 | 1.4711 | 479132 | 2137.55 |
| Patient 10 | 35 | 6 | 1.3158 | 1211180 | 2517.30 |
| Patient 10 | 7 | 1 | 4.9120 | 273713 | 1939.50 |
| Patient 10 | 35 | 6 | 1.6335 | 982682 | 2560.71 |
| Patient 10 | 24 | 2 | 1.9923 | 714959 | 2836.31 |
| Patient 10 | 42 | 3 | 3.1730 | 902932 | 3675.81 |
| Patient 10 | 2 | 0 | 92754290.9892 | 60348 | 2036.70 |
| Patient 10 | 18 | 0 | 1.4223 | 570071 | 2064.61 |
| Patient 10 | 13 | 1 | 2.0744 | 355778 | 2954.90 |
| Patient 10 | 14 | 1 | 2.0671 | 429142 | 2203.59 |
| Patient 10 | 12 | 0 | 1.3065 | 839278 | 2087.48 |
| Patient 10 | 34 | 3 | 2.7804 | 1338202 | 3116.21 |
| Patient 10 | 5 | 0 | 4.5452 | 96913 | 2279.62 |
| Patient 10 | 26 | 4 | 2.4351 | 840775 | 2123.24 |
| Patient 10 | 45 | 3 | 2.3016 | 1006837 | 3171.83 |
| Patient 10 | 17 | 2 | 3.2856 | 518375 | 2490.06 |
| Patient 10 | 5 | 0 | 11.1402 | 116094 | 3256.75 |
| Patient 10 | 10 | 1 | 2.1528 | 124892 | 2753.46 |
| Patient 10 | 17 | 3 | 1.5715 | 548622 | 2245.33 |
| Patient 10 | 22 | 0 | 1.5526 | 541007 | 1989.77 |
| Patient 10 | 25 | 3 | 1.5048 | 949193 | 2660.60 |
| Patient 10 | 14 | 2 | 2.7342 | 549074 | 2398.28 |
| Patient 10 | 27 | 3 | 3.8923 | 889867 | 2249.11 |
| Patient 10 | 12 | 0 | 1.2815 | 1074590 | 1663.67 |
| Patient 10 | 11 | 0 | 1.2572 | 258157 | 1665.92 |
| Patient 10 | 12 | 0 | 1.5121 | 579939 | 2063.68 |
| Patient 10 | 16 | 2 | 1.1334 | 604548 | 2292.17 |
| Patient 10 | 8 | 0 | 1.1096 | 320249 | 2183.73 |
| Patient 10 | 21 | 4 | 1.3420 | 1186788 | 1744.92 |
| Patient 10 | 11 | 1 | 4.2765 | 287237 | 1973.47 |
| Patient 10 | 13 | 0 | 1.5103 | 538152 | 3054.40 |
| Patient 10 | 81 | 12 | 2.5046 | 1843614 | 3362.61 |
| Patient 10 | 18 | 2 | 0.6756 | 815092 | 2388.72 |
| Patient 10 | 22 | 3 | 2.7137 | 236080 | 2168.08 |
| Patient 10 | 14 | 1 | 1.9053 | 376078 | 1779.89 |
| Patient 10 | 23 | 3 | 1.2425 | 1044296 | 2451.11 |
| Patient 10 | 4 | 1 | 2.3846 | 198915 | 1214.22 |
| Patient 10 | 17 | 1 | 1.5810 | 1139196 | 2130.50 |
| Patient 10 | 13 | 2 | 1.9041 | 274647 | 2050.83 |
| Patient 10 | 5 | 0 | 9.3600 | 193255 | 1771.33 |
| Patient 10 | 27 | 1 | 1.7191 | 971125 | 2126.55 |
| Patient 10 | 16 | 1 | 3.0304 | 479441 | 2095.93 |
| Patient 10 | 12 | 0 | 1.1064 | 451963 | 2044.08 |
| Patient 10 | 8 | 0 | 3.8751 | 266723 | 2228.96 |
| Patient 10 | 28 | 0 | 1.2898 | 1569809 | 3038.36 |
| Patient 10 | 21 | 1 | 1.0176 | 860119 | 2033.27 |
| Patient 10 | 12 | 0 | 1.5059 | 320460 | 1976.88 |
| Patient 10 | 23 | 3 | 2.4414 | 614827 | 2494.81 |

### REFERENCES

1. Hallek M, Bergsagel P L, Anderson K C. Multiple myeloma: increasing evidence for a multistep transformation process. Blood. 1998 Jan. 1; 91(1):3-21.
2. Dhodapkar M V1. MGUS to myeloma: a mysterious gammopathy of underexplored significance. Blood. 2016 Dec. 8; 128(23):2599-2606.
3. Albagoush S A, Azevedo A M. Cancer, Multiple Myeloma. StatPearls [Internet]. Treasure Island (FL): StatPearls Publishing; 2019 January-2019 Mar. 19. [BOOK}
4. Semin Oncol. 2019 April; 46(2):112-120. doi: 10.1053/j.seminoncol.2019.03.001. Epub 2019 Apr. 24. State of the science in smoldering myeloma: Should we be treating in the clinic?Hill E1, Dew A2, Kazandjian D3.
5. Kyle R A, Remstein E D, Therneau™, et al: Clinical course and prognosis of smoldering (asymptomatic) multiple myeloma. N Engl J Med 2007; 356: pp. 2582-2590.
6. Ziogas D C1, Dimopoulos M A1, Kastritis E1. Expert Rev Hematol. 2018 November; 11(11):863-879. doi: 10.1080/17474086.2018.1537776. Epub 2018 Oct. 26. Prognostic factors for multiple myeloma in the era of novel therapies.
7. Greipp, P. R., San Miguel, J., Durie, B. G., Crowley, J. J., Barlogie, B., Blade, J., Boccadoro, M., Child, J. A., Avet-Loiseau, H., Kyle, R. A., Lahuerta, J. J., Ludwig, H., Morgan, G., Powles, R., Shimizu, K., Shustik, C., Sonneveld, P., Tosi, P., Turesson, I. & Westin, J. (2005) International staging system for multiple myeloma. Journal of Clinical Oncology, 23, 3412-3420.
8. Cagnetta A1, Lovera D1, Grasso R1, Colombo N1, Canepa L1, Ballerini F1, Calvio M1, Miglino M1, Gobbi M1, Lemoli R1, Cea M1. Mechanisms and Clinical Applications of Genome Instability in Multiple Myeloma. Biomed Res Int. 2015; 2015:943096. doi: 10.1155/2015/943096. Epub 2015 Oct. 22.
9. Bolli N., Avet-Loiseau H., Wedge D. C., et al. Heterogeneity of genomic evolution and mutational profiles in multiple myeloma. Nature Communications. 2014; 5, article 2997 doi: 10.1038/ncomms3997.
10. Neri P., Bahlis N. J. Genomic instability in multiple myeloma: mechanisms and therapeutic implications. Expert Opinion on Biological Therapy. 2013; 13(supplement 1):S69-S82. doi: 10.1517/14712598.2013.814637.
11. Morgan G. J., Walker B. A., Davies F. E. The genetic architecture of multiple myeloma. Nature Reviews Cancer. 2012; 12(5):335-348. doi: 10.1038/nrc3257.
12. Gadji M1,2, Pozzo A R1. From cellular morphology to molecular and epigenetic anomalies of myelodysplastic syndromes. Genes Chromosomes Cancer. 2019 July; 58(7):474-483. doi: 10.1002/gcc.22689. Epub 2018 Nov. 18
17. Yu P L I, Wang Y, Tammur P, Tamm A, Punab M, Rangel-Pozzo A, Mai S. Distinct Nuclear Organization of Telomeresand Centromeres in Monoclonal Gammopathy of Undetermined Significance and Multiple Myeloma. Cells. 2019 Jul. 15; 8(7). pii: E723. doi: 10.3390/cells8070723.
18. Klewes L, Vallente R, Dupas E, Brand C, Grun D, Guffei A, Sathitruangsak C, Awe J A, Kuzyk A, Lichtensztejn D, Tammur P, Ilus T, Tamm A, Punab M, Rubinger M, Olujohungbe A, Mai S. Three-dimensional Nuclear Telomere Organization in Multiple Myeloma. Transl Oncol. 2013 Dec. 1; 6(6):749-56. eCollection 2013 Dec. 1.
19. Miyazaki K, Suzuki K. CD56 for Multiple Myeloma: Lack of CD56 May Be Associated with Worse Prognosis. Acta Haematol. 2018; 140(1):40-41. doi: 10.1159/000490231. Epub 2018 Aug. 15.
20. Schaefer, L. H.; Schuster, D.; Herz, H. Generalized approach for accelerated maximum likelihood based image restoration applied to three-dimensional fluorescence microscopy. Journal of microscopy 2001, 204, 99-107, DOI: 10.1046/j.1365-2818.2001.00949.x
21. Jones C H1, Pepper C, Baird D M. Telomere dysfunction and its role in haematological cancer. Br J Haematol. 2012 March; 156(5):573-87. doi: 10.1111/j.1365-2141.2011.09022.x. Epub 2012 Jan. 11.
22. Wark L, Quon H, Ong A, Drachenberg D, Rangel-Pozzo A, Mai S. Long-Term Dynamics of Three Dimensional Telomere Profiles in Circulating Tumor Cells in High-Risk Prostate Cancer Patients Undergoing Androgen-Deprivation and Radiation Therapy. Cancers (Basel). 2019 Aug. 14; 11(8). pii: E1165. doi: 10.3390/cancers11081165.
23. Drachenberg D, Awe J A, Rangel Pozzo A, Saranchuk J, Mai S. Advancing Risk Assessment of Intermediate Risk Prostate Cancer Patients. Cancers (Basel). 2019 Jun. 20; 11(6). pii: E855. doi: 10.3390/cancers11060855.
24. Rangel-Pozzo A, Corrêa de Souza D, Schmid-Braz A T, de Azambuja A P, Ferraz-Aguiar T, Borgonovo T, Mai S. 3D Telomere Structure Analysis to DetectGenomic Instability and Cytogenetic Evolution in Myelodysplastic Syndromes. Cells. 2019 Apr. 2; 8(4). pii: E304. doi: 10.3390/cells8040304.
25. Mai S1, Garini Y. The significance of telomeric aggregates in the interphase nuclei of tumor cells. J Cell Biochem. 2006 Apr. 1; 97(5):904-15.
26. Vermolen, B. J.; Garini, Y.; Mai, S.; Mougey, V.; Fest, T.; Chuang, T. C.-Y.; . . . Young, I. T. Characterizing the three-dimensional organization of telomeres. Cytometry Part A 2005, 67: 144-150, DOI: 10.1002/cyto.a.20159
27. Wu K D1, Orme L M, Shaughnessy J Jr, Jacobson J, Barlogie B, Moore M A. Telomerase and telomere length in multiple myeloma: correlations with disease heterogeneity, cytogenetic status, and overall survival. Blood. 2003 Jun. 15; 101(12):4982-9. Epub 2003 Feb. 27.
28. Cottliar A1, Pedrazzini E, Corrado C, Engelberger M I, Narbaitz M, Slavutsky I. Telomere shortening in patients with plasma cell disorders. Eur J Haematol. 2003 November; 71(5):334-40.
29. Walker B A #1, Wardell C P #1, Melchor L1, Brioli A1,2, Johnson D C1, Kaiser M F1, Mirabella F1, Lopez-Corral L3, Humphray S4, Murray L4, Ross M4, Bentley D4, Gutiérrez N C3, Garcia-Sanz R3, San Miguel J3, Davies F E5, Gonzalez D1, Morgan G J1. Intraclonal heterogeneity is a critical early event in the development of myeloma and precedes the development of clinical symptoms. Leukemia. 2014 February; 28(2):384-390. doi: 10.1038/leu.2013.199. Epub 2013 Jul. 2.
38. Padala S A, Barsouk A, Barsouk A, Rawla P, Vakiti A, Kolhe R, Kota V, Ajebo G H. Epidemiology, Staging, and Management of Multiple Myeloma. Med Sci (Basel). 2021 Jan. 20; 9(1):3. doi: 10.3390/medsci9010003. PMID: 33498356; PMCID: PMC7838784.
39. Rajkumar S V, Kumar S, Lonial S, Mateos M V. Smoldering multiple myeloma current treatment algorithms. Blood Cancer J. 2022 Sep. 5; 12(9):129. doi: 10.1038/s41408-022-00719-0.
40. Lussier T, Schoebe N, Mai S. Risk Stratification and Treatment in Smoldering Multiple Myeloma. Cells. 2021 Dec. 31; 11(1):130. doi: 10.3390/cells11010130.
41. Mateos M V, Hernendez M T, Salvador C, de la Rubia J, de Arriba F, Lucia López-Corral L, Rosiñol L, Paiva B, Palomera L, Bargay J, Oriol A, Prosper F, López J, Arguinano J M, Blade J, Lahuerta J J, San-Miguel J. Lenalidomide-dexamethasone versus observation in high-risk smoldering myeloma after 12 years of median follow-up time: A randomized, open-label study. Eur J Cancer. 2022 October; 174:243-250. DOI: 10.1016/j.ejca.2022.07.030.
42. Mathur S, Glogowska A, McAvoy E, Righolt C, Rutherford J, Willing C, Banik U, Ruthirakuhan M, Mai S, Garcia A. Three-dimensional quantitative imaging of telomeres in buccal cells identifies mild, moderate, and severe Alzheimer's disease patients. J Alzheimers Dis. 2014. 39(1):35-48. doi: 10.3233/JAD-130866.
43. Rangel-Pozzo A, Yu P L I, Lal S, Asbaghi Y, Sisdelli L, Tammur P, Tamm A, Punab M, Klewes L, Louis S, Knecht H, Olujoi-lungbe A, Mai S, Telomere Architecture Correlates with Aggressiveness in Multiple Myeloma. Cancers (Basel). 19 Apr. 2021 (19 Apr. 2021), Vol. 13(8), pp. 1969-1985.
44. Rajkumar S V et al., International Myeloma Working Group updated criteria for the diagnosis of multiple myeloma, Lancet Oncol. 2014, 5(12):e538-48.

1. A method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; and
optionally providing the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with stable SMM is not likely to progress to MM within 2 years.

2. The method of claim 1, wherein the test sample is a bone marrow sample or a blood sample, optionally a diagnostic bone marrow biopsy sample or a peripheral liquid biopsy blood sample.

3. The method of claim 1 or 2, wherein the prognosis or the diagnosis is provided to the subject or the subject's medical professional, optionally at time of SMM diagnosis.

4. The method of any one of claims 1 to 3, the assaying comprising:
labelling nuclei of the plurality of the plasma cells with a fluorescent nuclear stain or probe, optionally wherein the fluorescent nuclear stain is 4′,6-diamidino-2-phenylindole (DAPI);
tagging telomeres in the plurality of plasma cells through in situ hybridization with a telomere-specific labelled probe, optionally a peptide nucleic acid (PNA) probe,
mounting the test sample using an antifade mounting medium;
3D imaging the test sample; and
measuring on the 3D images values for the telomere parameters to obtain the 3D telomere organization sample signature.

5. The method of claim 4, wherein assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

6. The method of claim 4 or 5, wherein the 3D imaging comprises acquiring an image dataset of different planes of 3D q-FISH fluorescent signals and reconstructing a 3D image of the telomeres using deconvolution of the images performed with a constrained iterative algorithm, optionally using fluorescence microscopy and/or obtaining a stack of at least 50 images with a sample distance of 200 nm along a z direction and 102 nm in each of a x and a y direction.

7. The method of any one of claims 1 to 6, wherein the 3D telomere organization sample signature is determined from interphase plasma cells.

8. The method of any one of claims 1 to 7, wherein the telomere parameter (each or all) comprises an absolute value, a mean, a median, a ratio, a percentile, a quartile, a rank, a range (optionally a percentile range or a quartile range), or a combination thereof.

9. The method of any one of claims 1 to 8, wherein the sample is a diagnostic sample.

10. The method of any one of claims 1 to 9, wherein the one or more of the telomere parameter of the classification model is selected to have an accuracy of at least 70%, at least 75%, at least 80%, at least 85%, at least 90%, at least 95%, at least 96%, at least 97%, least 98%, at least 99% or 100% in distinguishing between stable SMM and high-risk SMM.

11. The method of any one of claims 1 to 10, wherein the subject is a human subject.

12. The method of any one of claims 1 to 11, wherein when the subject is prognosed or diagnosed as having high-risk SMM, the subject is subsequently treated with one or more of lenalidomide, dexamethasone, siltuximab, daratumumab, bortezomib, elotuzumab, carfilzomib, thalidomide, cyclophosphamide and combinations thereof.

13. The method of claim 1 to 12, wherein the subject prognosed or diagnosed as having high-risk SMM is subsequently treated with: a) bortezomib and dexamethasone; b) siltuximab, c) daratumumab, lenalidomide, bortezomib and dexamethasone, d) elotuzumab, carfilzomib, lenalidomide, daratumumab, and dexamethasone, optionally for 3 to 4 cycles as induction therapy, e) bortezomib, lenalidomide and dexamethasone, f) bortezomib, thalidomide and dexamethasone, g) bortezomib, cyclophosphamide and dexamethasone, h) lenalidomide, or i) lenalidomide and dexamethasone.

14. The method of claim 1 to 13, wherein when the subject is prognosed or diagnosed as having stable SMM or as having high-risk SMM, the subject is subsequently monitored.

15. In an embodiment, the method of monitoring a subject prognosed or diagnosed as having stable SMM or as having high-risk SMM comprises:
obtaining a subsequent sample from the subject, the subsequent sample comprising a plurality of plasma cells;
assaying the plurality of plasma cells according to any assaying step described herein, to obtain a 3D telomere organization monitoring signature;
applying a classification model to the 3D telomere organization monitoring signature to obtain an output classification of stable SMM or high-risk SMM, the classification model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates;
comparing the output classification of the 3D telomere organization monitoring signature to an output classification of a previous sample; and
providing an updated clinical outcome prognosis or an updated diagnosis.

16. The method of any one of claims 1 to 14, wherein the method first comprises obtaining the test sample from the patient.

17. A method of treating a subject with smoldering multiple myeloma (SMM) based on a 3D telomere organization signature from the subject, comprising administering to the subject a treatment selected from claim 12 or 13 when the subject has high-risk SMM, or monitoring the subject when the subject has stable SMM.

18. A method of providing a personalized treatment plan for a subject with smoldering multiple myeloma (SMM) based on a 3D telomere organization sample signature of the subject, comprising providing the subject the personalized treatment plan to be administered to the subject when the subject has high-risk SMM or monitoring the subject when the subject has stable SMM determined according to the method of any one of claims 1 to 11, wherein the treatment plan comprises a treatment selected from any one of claim 12 or 13.

19. The method of claim 17 or 18, wherein the subject having high-risk SMM or the subject having stable SMM is prognosed or diagnosed as having high-risk SMM or stable SMM according to the method of any one of claims 1 to 12 or 15.

20. Use of the method according to any one of claims 1 to 11 or 15 for treating a subject with SMM.

21. A prognosis or diagnosis determined using the method according to any one of claims 1 to 11 or 18 for use in treating a subject with SMM.

22. The use of claim 20 or the prognosis or diagnosis for treating of claim 21, wherein treating comprises administering to the subject a treatment selected from any one of claims 20 to 30 when the subject has high-risk SMM, or monitoring the subject when the subject has stable SMM.

23. An assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising telomere parameters: a) nuclear telomere distribution, a/c ratio, and total telomere length; b) a/c ratio and telomere numbers; c) a/c ratio and nuclear telomere distribution, or d) a/c ratio and telomere aggregates; applying a classification model to the 3D telomere organization sample signature to obtain an output classification of stable SMM or high-risk SMM, the model being a model trained to distinguish between stable SMM and high-risk SMM and consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; providing the clinical outcome prognosis or the diagnosis according to the output classification, the clinical outcome prognosis or the diagnosis being of stable SMM or of high-risk SMM, wherein the subject with high-risk SMM is likely to progress to multiple myeloma (MM) within 2 years and the subject with stable SMM is not likely to progress to MM within 2 years; and selecting a therapy according to claim 12 or 13 for the subject when the subject is identified as having high-risk SMM, or monitoring the subject when the subject is identified as having stable SMM.
